(19) United States
(12) Patent Application Publication
(10) Pub. No.: US 2018/0134680 A1
(43) Pub. Date: May 17, 2018

(54) METHOD FOR PRODUCING CYCLIC ESTERS

(71) Applicant: BASF SE, Ludwigshafen (DE)

(72) Inventors: Wolfgang SIEGEL, Limburgerhof (DE); Karin SCHEIN-ALBRECHT, Worms (DE); Ralf PELZER, Fürstenberg (DE)

(21) Appl. No.: 15/577,590

(22) PCT Filed: May 27, 2016
(86) PCT No.: PCT/EP2016/061963

§ 371 Date: Nov. 28, 2017

(30) Foreign Application Priority Data
May 29, 2015 (EP) 15169797.6

(51) Int. Cl. C07D 313/00

(52) U.S. Cl. CPC C07D 313/00

(57) ABSTRACT

The invention relates to a method for producing cyclic esters of general formula (I.a) or (I.b) in the presence of at least one high-boiling metal alkoxide catalyst. The invention further relates to the stereoisomers of 18-methyl-1-oxacyclooctadec-10-en-2-one and to the use thereof as an odorous substance and/or flavoring substance, to compositions that contain at least one of the stereoisomers of 18-methyl-1-oxacyclooctadec-10-en-2-one and additionally a carrier material, to fragrance compositions and/or to fragrant-substance materials that contain at least one of these compounds, and to a method for giving an odor or flavor to or changing an odor or flavor of compositions by adding at least one of the mentioned compounds to said compositions.

The present invention relates to a process for the preparation of cyclic esters in the presence of at least one high-boiling metal alkoxide catalyst. The invention likewise relates to the stereoisomers of 18-methyl-1-oxacyclooctadec-10-en-2-one and the use thereof as fragrance and/or flavoring, to compositions which comprise at least one of the stereoisomers of 18-methyl-1-oxacyclooctadec-10-en-2-one and additionally a carrier material, to scent compositions and/or fragrance materials which comprise at least one of these compounds, and to a method for imparting or altering an odor or taste of compositions by adding at least one of the specified compounds to these compositions.

### BACKGROUND OF THE INVENTION

Macrocyclic compounds which have a musk-like odor have been valued aroma chemicals in the fragrance industry for a long time. These compounds include both macrocyclic ketones such as e.g. cyclopentadecanone (Exalton®) or (Z)-9-cyclo-heptadecen-1-one (zibetone) as well as macrocyclic esters or diesters such as, for example, oxacyclohexadecan-2-one (Exaltolid®) (1), oxacycloheptadecan-2-one (hexadecanolide) (2), 1,4-dioxacycloheptadecane-5,17-dione (ethylene brassylate) (3) or oxacycloheptadec-10-en-2-one (ambrettolide) (4), and further functionalized macrocycles.

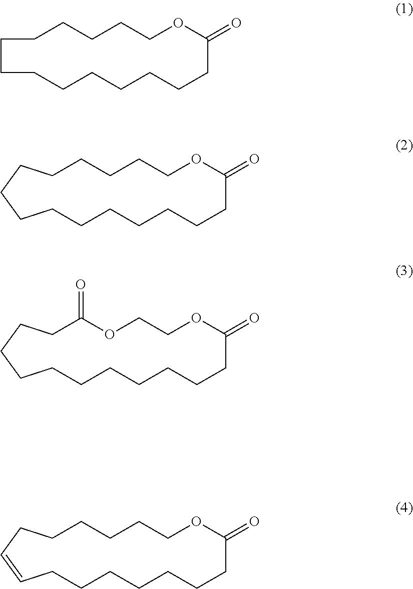 [SMILES: O=C1CCCCCCCCCCCCCCO1 ; O=C1CCCCCCCCCCCCCCCO1 ; O=C1CCCCCCCCCCCC(=O)OCCO1 ; O=C1CCCCCCC/C=C\CCCCCCO1]

Isolating these fragrances from natural sources is in most cases very expensive and the amounts that can be obtained this way are limited. Moreover, the purity or production amount of these fragrances often varies on account of changeable environmental conditions during the production of the raw materials from which these are isolated.

There is therefore a need for effective processes for the preparation of cyclic compounds based on medium, and specifically based on large, rings which have at least one keto or ester group. Medium rings generally have 8 to 11 carbon atoms, and above 12 carbon atoms the rings are described as large. Compounds based on large rings are also referred to as macrocyclic compounds.

The known processes for the preparation of macrocyclic compounds with advantageous organoleptic properties are mostly based on complex, multistage syntheses and are therefore laborious and expensive. An overview of the synthesis of macrocyclic musk compounds is given for example by A. S. Williams in Synthesis 1999, 10, 1707-1723.

Macrocyclic esters can be prepared inter alia by cyclization of the corresponding hydroxycarboxylic acids or hydroxycarboxylic acid esters or by reaction of the corresponding diacids or diacid esters with diols. For this, many methods are known in the prior art.

For example, Parenty et al., Chem. Rev., 2006, volume 106, pages 911-939, describe a method for the synthesis of macrocyclic esters in which firstly the respective acid or alcohol function of the feed materials is activated. Then, the thus activated feed materials are cyclized to give the macrocyclic esters. However, high yields can only be achieved with this process if the activated feed materials are present in highly diluted form during the cyclization. This limitation generally also applies to other processes known in the prior art which include an intramolecular esterification reaction. Such processes are therefore mostly unsuited for use in the industrial sector.

When producing macrocyclic lactones on an industrial scale, a different principle is generally followed. Here, the feed materials are firstly converted to oligomeric or polymeric esters, which are then depolymerized at temperatures in the range from 200 to 350° C. and low pressures of below 100 mbar in the presence of typical transesterification catalysts. In this process, the monomeric cyclic ester that is formed in the equilibrium is continuously distilled off from the reaction mixture as the lowest boiling component.

JP 55002640, for example, describes a process for the preparation of macrocyclic esters in which linear polyesters which have been obtained by the condensation of hydroxycarboxylic acids or of diacids with glycols are depolymerized and cyclized in the presence of titanium alkoxide catalysts.

EP 1097930 describes a process for preparing macrocyclic lactones, in which a hydroxycarboxylic ester is subjected to an intramolecular transesterification, wherein the ester group of the hydroxycarboxylic ester is an alkyl group or an alkylene oxide oligomer. It is stated that this reaction proceeds particularly advantageously in the presence of an alcohol selected from aliphatic alcohols and polyalkylene oxide alcohols.

EP 0940396 describes a process for preparing lactones proceeding from omega-hydroxycarboxylic acids or esters thereof in monomeric, oligomeric or polymeric form. One operation described therein is addition of high-boiling polyalkylene glycol diethers to the reaction medium.

As a rule, the hydroxyl and/or carboxy fatty acids required for the polymerization and cyclization are only accessible synthetically with difficulty. It is known in the prior art that these can be isolated from sophorolipids produced by fermentation and can be used advantageously as feed materials with the synthesis of macrocyclic esters.

CH 430679 describes a process for the preparation of oxacyloheptadecan-2-one (hexadecanolid) and 16-methyl-oxacycloheptadecan-2-one from a mixture of 15- and 16-hydroxypalmitic acid. The 15- or 16-hydroxypalmitic acid is obtained here from sophorolipids which are formed during the fermentative reaction of palmitic acid by Candida magnoliae.

DE 2834117 describes a process for the preparation of an ester of a hydroxy fatty acid and a mono- or polyhydric alcohol, where the hydroxy fatty acid starting material used is sophorolipids which are formed during the fermentation of fatty acids with Candida bombicola. The esters can be cyclized to give large lactone rings which have a musk scent.

In the processes that can be used industrially that are known in the prior art, high-viscosity bottoms are generally formed during or after the polymerization step. So that the reaction medium remains stirrable during the subsequent depolymerization and the monomeric cyclization product can be distilled more easily from the high-viscosity medium, it has proven to be advantageous to use a high-boiling solvent. Such high-boiling solvents are also referred to as “bottoms diluents”.

JP 55120581, for example, describes a process for the preparation of macrocyclic esters by depolymerization and cyclization of linear polyesters, in which polyalkylene glycols, polyalkylene glycol esters, monobasic carboxylic acids, carboxylic acid esters, carboxylic anhydrides, alcohols or alcohol esters are added as viscosity-reducing additives to the reaction mixture.

DE 3225431 A1 describes a process for the preparation of macrocyclic ester compounds by decomposition and cyclization of linear ester compounds, in which a glycol and/or an oligo-ester compound is used as solvent/bottoms diluent,

EP 0260680 describes a process for the preparation of macrocyclic esters by the catalyzed thermal depolymerization of linear polyesters in which an olefinic polymer, which is inert under the described reaction conditions and is present in liquid form, is used as solvent. Specifically, polyethylene is used as the olefinic polymer.

WO 02/16345 describes a process for the preparation of macrocyclic esters by the thermal cleavage of linear oligoesters in the presence of thermally stable benzene derivatives as solvents.

EP 0739889 A1 describes a two-stage process for the preparation of macrocyclic compounds in which difunctional feed materials are condensed in a first step under autocatalysis or in the presence of Bronsted acids to give oligomers. The oligomers obtained in this way are depolymerized in a second step in the presence of Lewis acids, the solvents or bottoms diluents used being polyalkylene glycol dialkyl ethers which have a molecular weight of 500-3000 Da. Specifically described is the polycondensation of 15-hydroxypentadecanoic acid, and of dodecanedioic acid or tridecanedioic acid methyl ester with ethylene glycol to the corresponding linear oligoesters, where the condensation takes place autocatalytically or in the presence of Bronsted acids, such as p-toluenesulfonic acid, and the water which forms is removed by distillation. The oligoesters are then converted to the cyclic mono- or diesters under addition of polyethylene glycol dimethyl ethers, with a molecular weight of 2000 Da, in the presence of tin-comprising catalysts or in the presence of tetrabutyl titanate. It is explicitly disclosed here that the use of polyether glycols and/or polyether glycol esters as bottoms diluent in the depolymerization step is unsuitable since these participate in the polymerization in an undesired manner, which leads to considerable yield losses.

In most of the industrial processes known in the prior art, the catalyst used in the transesterification or depolymerization step co-distills off at least partially during the distillation of the monomeric cyclization product and later has to be separated off from the product in a laborious manner.

The choice of solvent used as bottoms diluent is also critical since this co-distills off under certain circumstances during the distillation of the monomeric cyclization product and in so doing can entrain the catalyst located in the bottom and/or otherwise adversely affect the activity of the catalyst.

### SUMMARY OF THE INVENTION

The object of the present invention is to provide an improved process for the preparation of macrocyclic esters, by means of which the aforementioned disadvantages can be avoided. In particular, the aim is to provide a catalyst which can be used advantageously for the polymerization and depolymerization and does not co-distill off from the reaction mixture during the distillative removal of the monomeric cyclization product. Nevertheless, good yields should be able to be achieved with the catalyst. The solvent used as bottoms diluent in the depolymerization step should moreover not adversely affect the activity of the catalyst.

This object is achieved by a process for the preparation of macrocyclic compounds of the general formulae (I.a) or (I.b)

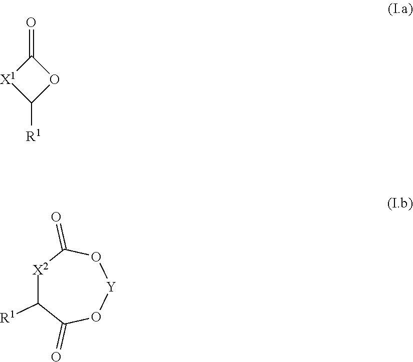

in which
X¹ is an unbranched or branched C₄-C₃₀-alkylene group or an unbranched or branched 04-C₃₀-alkenylene group, comprising 1, 2 or 3 double bonds,
X² is an unbranched or branched C₁-C₃₀-alkylene group or an unbranched or branched C₂-C₃₀-alkenylene group, comprising 1, 2 or 3 double bonds,
Y is an unbranched or branched C₂-C₁₀-alkylene group and
R¹ is hydrogen or an unbranched or branched C₁-C₁₀-alkyl group,

in which
a) at least one compound of the general formula (II.a) or (II.b)

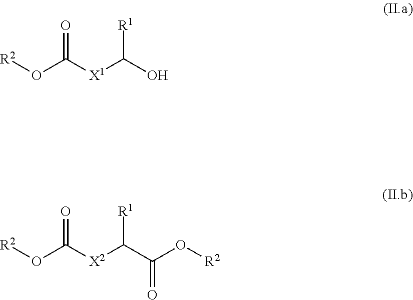

is provided, in which
X¹, X² and R¹ have the meanings given above and
R² is hydrogen or an unbranched or branched C₁-C₃₀-alkyl group,
b.1) the at least one compound (II.a) is reacted in the presence of at least one catalyst which is selected from metal alkoxides, and also in the presence of at least one polyether compound (PE) with a number-average molecular weight of at least 200 g/mol, to give a reaction mixture which comprises at least one macrocyclic compound of the general formula (I.a),

or
b.2) the at least one compound (II.b) is reacted in the presence of at least one catalyst which is selected from metal alkoxides, and also in the presence of at least one polyether compound (PE) with a number-average molecular weight of at least 200 g/mol, and additionally in the presence of at least one diol HO—Y—OH, where Y has the meaning given above, to give a reaction mixture which comprises at least one macrocyclic compound of the general formula (I.b),

where a product stream enriched in the macrocyclic compounds of the general formula (I.a) or (I.b) is removed by distillation from the reaction mixture obtained in step b.1) or b.2), and a bottom product enriched in the polyether compound (PE) and the catalyst is obtained.

Furthermore, it has been found that the stereoisomers of 18-methyl-1-oxacyclooctadec-10-en-2-one prepared by the process according to the invention, i.e. the compounds (10Z,18S)-18-methyl-1-oxacyclooctadec-10-en-2-one, (10Z,18R)-18-methyl-1-oxacyclooctadec-10-en-2-one, (10E,18S)-18-methyl-1-oxacyclooctadec-10-en-2-one, and (10E,18R)-18-methyl-1-oxacyclooctadec-10-en-2-one, have a musk-like odor. These compounds are novel and their suitability as fragrances and/or flavorings has therefore likewise not been described.

Consequently, the present invention relates to the compounds (10Z,18S)-18-methyl-1-oxacyclooctadec-10-en-2-one, (10Z,18R)-18-methyl-1-oxacyclooctadec-10-en-2-one, (10E,18S)-18-methyl-1-oxacyclooctadec-10-en-2-one, and (10E,18R)-18-methyl-1-oxacyclooctadec-10-en-2-one.

Furthermore, the present invention relates to the use of (10Z,18S)-18-methyl-1-oxacyclooctadec-10-en-2-one, (10Z,18R)-18-methyl-1-oxacyclooctadec-10-en-2-one, (10E,18S)-18-methyl-1-oxacyclooctadec-10-en-2-one, and (10E,18R)-18-methyl-1-oxacyclooctadec-10-en-2-one fragrances and/or flavorings.

Furthermore, the present invention relates to the use of at least one of the aforementioned compounds as constituent of a composition which additionally comprises a carrier material, where the composition is selected from detergents, laundry care compositions, cleaners, cosmetic preparations, fragrance-containing hygiene articles, foods, food supplements, air fresheners, perfumes, pharmaceutical preparations and crop protection agents.

Furthermore, the present invention relates to a scent composition and/or a fragrance material comprising at least one of the aforementioned compounds and a carrier material.

Furthermore, the present invention relates to a method for imparting or altering an odor or taste of a composition, in which at least one of the aforementioned compounds is added to the composition in an amount which imparts an odor or taste to the composition or alters the odor or taste of the composition.

### EMBODIMENTS OF THE INVENTION

The invention specifically comprises the following preferred embodiments:
1. A process for the preparation of macrocyclic compounds of the general formula (I.a) or (I.b)

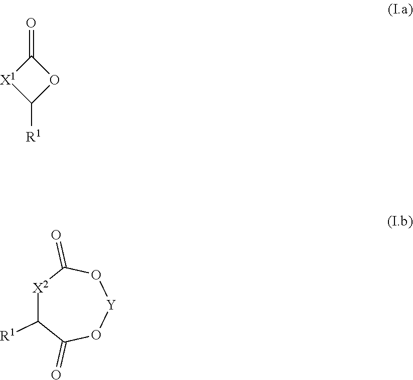

in which
X¹ is an unbranched or branched C₄-C₃₀-alkylene group or an unbranched or branched C₄-C₃₀-alkenylene group, comprising 1, 2 or 3 double bonds,
X² is an unbranched or branched C₁-C₃₀-alkylene group or an unbranched or branched C₂-C₃₀-alkenylene group, comprising 1, 2 or 3 double bonds,
Y is an unbranched or branched C₂-C₁₀-alkylene group and
R¹ is hydrogen or an unbranched or branched C₁-C₁₀-alkyl group,
in which
a) at least one compound of the general formula (II.a) or (II.b)

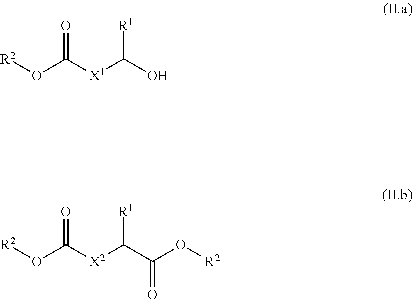

is provided, in which
X¹, X² and R¹ have the meanings given above and
R² is hydrogen or an unbranched or branched C₁-C₃₀-alkyl group,
b.1) the at least one compound (II.a) is reacted in the presence of at least one catalyst which is selected from metal alkoxides, and also in the presence of at least one polyether compound (PE) with a number-average molecular weight of at least 200 g/mol, to give a reaction mixture which comprises at least one macrocyclic compound of the general formula (I.a),
or
b.2) the at least one compound (II.b) is reacted in the presence of at least one catalyst which is selected from metal alkoxides, and also in the presence of at least one polyether compound (PE) with a number-average molecular weight of at least 200 g/mol, and additionally in the presence of at least one diol HO—Y—OH, where Y has the meaning given above, to give a reaction mixture which comprises at least one macrocyclic compound of the general formula (I.b),
where a product stream enriched in the macrocyclic compounds of the general formula (I.a) or (I.b) is removed by distillation from the reaction mixture obtained in step b.1) or b.2), and a bottom product enriched in the polyether compound (PE) and the catalyst is obtained.
2. The process according to embodiment 1, where the at least one catalyst used in step b.1) or b.2) has a boiling point at 5 mbar of more than 250° C.
3. The process according to either of embodiments 1 and 2, where, for the distillative removal of the product stream enriched in the compounds (I.a) or (I.b), at least one solvent (S) different from the polyether compound (PE) is added as entrainer to the reaction mixture obtained in step b.1) or b.2) and/or an inert gas stream is fed into the reaction mixture.
4. The process according to any one of embodiments 1 to 3, where the distillative removal of the product stream enriched in the compounds (I.1) or (I.b) takes place after the reaction in step b.1) or b.2).
5. The process according to any one of the preceding embodiments, where, in the compounds of the general formulae (I.a) and (I.b), the radical X¹ has 11 to 21 ring carbon atoms and the radicals X² and Y together have 9 to 19 ring carbon atoms.
6. The process according to any one of the preceding embodiments, where, in the compounds of the general formulae (I.a), (I.b), (II.a) and (II.b),
   R¹ is hydrogen or methyl,
   X¹ is an unbranched C₁₂-C₁₆-alkylene group or an unbranched C₁₂-C₁₆-alkenylene group, comprising a double bond,
   X² is an unbranched C₉-C₁₃-alkylene group or an unbranched alkenylene group, comprising a double bond,
   Y is an unbranched C₂-C₄-alkylene group and
   R² is hydrogen or an unbranched C₁-C4-alkyl group,
   where the radicals X² and Y together have 11 to 15 directly bridging carbon atoms.
7. The process according to any one of the preceding embodiments, where the distillatively removed product stream enriched in the compounds (I.a) or (I.b) comprises at least some of the solvent (S) and optionally additionally some of the polyether compound (PE) and the product stream is subjected to a separation to give a fraction enriched in the solvent (S) and optionally in the polyether compound (PE) and a product fraction which comprises predominantly macrocyclic compounds of the general formula (I.a) or (I.b).
8. The process according to embodiment 7, where the product fraction is subjected to a further purification, preferably a distillation.
9. The process according to embodiments 7 and 8, where the fraction enriched in the solvent (S) and optionally in the polyether compound (PE) is returned again to the reaction in step b.1) or b.2).
10. The process according to any one of the preceding embodiments, where the steps b.1) or b.2) and the distillative removal of the product stream enriched in the compounds (I.a) or (I.b) are carried out continuously.
11. The process according to any one of the preceding embodiments, where the at least one polyether compound (PE) is selected from compounds of the general formula (III)

R³—O—[Z—O]ₙ—R³ (III)
in which
Z is selected independently of the others from ethylene, 1,2-propylene, 1,3-propylene, 1,2-butylene, 2,3-butylene and 1,4-butylene,
n is an integer from 3 to 250 and
either one radical R³ is hydrogen and the other radical R³ is C₁-C₃₀-alkyl, or one radical R³ is hydrogen and the other radical R³ is —(C═O)—(C₁-C₃₀-alkyl),
or both radicals R³ are hydrogen,
or both radicals R³ are —(C═O)—(C₁-C₃₀-alkyl).
12. The process according to any one of the preceding embodiments, wherein the at least one polyether compound (PE) is selected from compounds of the general formula (III) in which
   Z and n are as defined above, and
   one of the radicals R³ is hydrogen, and
   the other radical R³ is hydrogen or is C₁-C₁₀-alkyl or is —(C═O)—C₁-C₃₀-alkyl).
13. The process according to either of embodiments 11 and 12, where
   one radical R³ is hydrogen, and
   the other radical R³ is hydrogen or is C₁-C₁₀-alkyl or is —(C═O)—(C₁-C₁₀-alkyl),
14. The process according to any one of the preceding embodiments, where the at least one metal alkoxide catalyst used in step b.1) or b.2) is prepared by reacting at least one metal compound, selected from metal oxides, alkyl metal oxides, metal salts or metal alkoxides of the general formula M[O(C₁-C₄-alkyl)]ₘ, where m has the values 1, 2, 3 or 4, with at least one polyether compound (PE), as defined in embodiment 1 or 11.
15. The process according to embodiment 14, where the preparation of the at least one metal alkoxide catalyst takes place in situ.
16. The process according to embodiment 14, where the preparation of the at least one metal alkoxide catalyst takes place before step b.1) or b.2).
17. The process according to any one of embodiments 14 to 16, where the low-boiling components optionally forming during the preparation of the at least one metal alkoxide catalyst are removed by distillation.
18. The process according to embodiment 17, where, for the distillative removal of the low-boiling components optionally formed during the preparation of the at least one metal alkoxide catalyst, a stream of inert gas is used.
19. The process according to any one of embodiments 14 to 18, where the metal of the metal oxide, alkyl metal oxide, metal salt or metal alkoxide M[O(C₁-C4-alkyl)]ₘ used for the preparation of the at least one metal alkoxide catalyst is selected from alkali metals, alkaline earth metals, transition metals of the 4th, 7th, 8th, 9th and 12th group, and metals and/or semi-metals of the 13th, 14th and 15th group of the Periodic Table of the Elements.
20. The process according to any one of embodiments 1 to 14 and 16 to 19, where the preparation of the at least one metal alkoxide catalyst takes place in the absence of the feed materials (I.a), (I.b) and of the diol HO—Y—OH.
21. The process according to any one of the preceding embodiments, where the bottom product enriched in step c) in the polyether compound (PE) and the catalyst is recycled to further reaction in step b.1) or b.2).
22. The process according to any one of the preceding embodiments, where the at least one solvent (S) is selected from C₂-C₁₅-alkanols, glycerol, pentaerythritol, C₂-C₄-alkylene glycols and mono- and di-(C₁-C₄-alkyl) ethers thereof, polyalkylene glycols different from the compounds PE and the mono- and dialkyl ethers thereof, aromatic hydrocarbons and mixtures thereof.
23. The process according to any one of the preceding embodiments, where the provision of the compounds (II.a) or (II.b) comprises the following steps
   a.1) provision of a C₆-C₂₂-carboxylic acid,
   a.2) conversion of the C₆-C₂₂-carboxylic acid provided in step a.1) to omega- and/or (omega-1)-hydroxylated or omega-carboxylated C₆-C₂₂-carboxylic acids,
   a.3) optionally the oxidation of the omega-hydroxylated C₆-C₂₂-carboxylic acids obtained in step a.2) to the corresponding omega-carboxylated C₆-C₂₂-carboxylic acids,
   a.4) optionally the esterification of the omega- and/or (omega-1)-hydroxylated C₆-C₂₂-carboxylic acids from step a.2) or of the carboxylated C₆-C₂₂-carboxylic acids from steps a.2) or a.3) with unbranched or branched C₁-C₆-alkanols.
24. The process according to embodiment 22, where the hydroxylation or carboxylation in step a.2) is carried out by fermentation.
25. A compound (I.a) selected from (10Z,18S)-18-methyl-1-oxacyclooctadec-10-en-2-one, (10Z,18R)-18-methyl-1-oxacyclooctadec-10-en-2-one, (10E,18S)-18-methyl-1-oxacyclooctadec-10-en-2-one, and (10E,18R)-18-methyl-1-oxacyclo-octadec-10-en-2-one.
26. The use of at least one of the compounds specified in embodiment 24 as fragrance and/or flavoring.
27. The use according to embodiment 26, where the at least one compound is a constituent of a composition which additionally comprises a carrier material.
28. The use according to embodiment 27, where the composition is selected from detergents and cleaners, cosmetic preparations, fragrance-containing hygiene articles, foods, food supplements, air fresheners, perfumes, pharmaceutical preparations and crop protection agents.
29. A scent composition and/or a fragrance material comprising at least one of the compounds specified in embodiment 25 and a carrier material.
30. A method for imparting or altering an odor or taste of a composition, in which at least one of the compounds specified in embodiment 25 is added to the composition in an amount which imparts an odor or taste to the composition or alters the odor or taste of the composition.

The process according to the invention is characterized by the following advantageous properties:

By virtue of the simultaneous use of a bottoms diluent (polyether compound PE) and an entrainer (solvent S), the distillative removal of the macrocyclic ester from the reaction mixture can take place very rapidly and/or at high flow rates.

Even during a rapid removal, the high-boiling catalyst does not distill off together with the macrocyclic esters.

It is generally possible to dispense with using laborious measures for the purification of the macrocyclic esters obtained by the process according to the invention.

Consequently, the process according to the invention permits the preparation of macrocyclic esters in good yields and purities for relatively short reaction times, i.e. in high space-time yield,

The process according to the invention can be carried out continuously and is characterized by its simplicity and cost-effectiveness.

The catalyst used in the process according to the invention can be recycled when the reaction is complete for the preparation of further macrocyclic esters, or be stored for a prolonged time.

Feed materials that are relatively easy to access can be used in the process according to the invention. Short-chain feed materials can be commercially acquired or be synthesized without problem. Long-chain feed materials can be produced relatively easily from fatty acids by a fermentation method.

The isomers of 18-methyl-1-oxacyclooctadec-10-en-2-one that can be prepared with the process according to the invention are characterized by advantageous organoleptic properties, in particular by a musk-like odor. They can therefore be used advantageously as fragrance or flavoring or as a constituent of a scent composition and/or a fragrance material.

On account of their physical properties, the isomers of 18-methyl-1-oxacyclooctadec-10-en-2-one have very good, practically universal dissolution properties for other odorous substances or other commercially available ingredients, as are used in scent compositions, in particular in perfumes.

The isomers of 18-methyl-1-oxacyclooctadec-10-en-2-one are expected to have a very low toxicity since they belong to a group of compounds which appear to have no notable toxicity. Structurally very similar macrocyclic esters, such as oxacycloheptadec-10-en-2-one (ambrettolide), are already used as scents.

### DETAILED DESCRIPTION OF THE INVENTION

In the context of the present invention, the expression “C₁-C₃₀-alkyl” refers to unbranched alkyl groups having 1 to 30 carbon atoms or branched alkyl groups having 3 to 30 carbon atoms. Preferably, “C₁-C₃₀-alkyl” is unbranched alkyl groups having 1 to 20 carbon atoms or branched alkyl groups having 3 to 20 carbon atoms. These include, for example, methyl, ethyl, propyl, isopropyl, n-butyl, isobutyl, sec-butyl, tert-butyl, n-pentyl, 2-pentyl, 2-methylbutyl, 3-methylbutyl, 1,2-dimethylpropyl, 1,1-dimethylpropyl, 2,2-dimethylpropyl, 1-ethylpropyl, n-hexyl, 2-hexyl, 2-methylpentyl, 3-methylpentyl, 4-methylpentyl, 1,2-dimethylbutyl, 1,3-dimethylbutyl, 2,3-dimethylbutyl, 1,1-dimethylbutyl, 2,2-dimethylbutyl, 3,3-dimethylbutyl, 1,1,2-trimethylpropyl, 1,2,2-trimethylpropyl, 1-ethylbutyl, 2-ethylbutyl, 1-ethyl-2-methylpropyl, n-heptyl, 1-methylhexyl, 2-methylhexyl, 1-ethylpentyl, 2-ethylpentyl, 1-propylbutyl, 1-ethyl-2-methylbutyl, n-octyl, isooctyl, 2-ethylhexyl, n-nonyl, isononyl, 2-propylhexyl, n-decyl, isodecyl, 2-propylheptyl, n-undecyl, isoundecyl, n-dodecyl, isododecyl, n-tridecane, n-tetradecane, n-pentadecane, n-hexadecane, isohexadecane, n-heptadecane, n-octadecane, n-nonadecane, n-decosane and the like. C₁-C₃₀-Alkyl is particularly preferably unbranched C₁-C₁₀-alkyl groups or branched C₃-C₁₀-alkyl groups, in particular unbranched C₁-C₆-alkyl groups or branched C₃-C₆-alkyl groups. Specifically, C₁-C₃₀-alkyl is unbranched C₁-C₄-alkyl groups, very specifically methyl or ethyl.

The expression “C₁-C₃₀-alkyl” includes in its definition also the expressions “C₁-C₁₀-alkyl”, “C₁-C₆-alkyl” and “C₁-C₄-alkyl”.

In the context of the present invention, the expression “C₁-C₃₀-alkylene” refers to divalent hydrocarbon radicals having 1 to 30 carbon atoms. The divalent hydrocarbon radicals can be unbranched or branched. These include, for example, methylene, 1,2-ethylene, 1,2-propylene, 1,3-propylene, 1,2-butylene, 1,3-butylene, 1,4-butylene, 2-methyl-1,3-propylene, 1,5-pentylene, 2-methyl-1,4-butylene, 2,2-dimethyl-1,3-propylene, 1,6-hexylene, 2-methyl-1,5-pentylene, 3-methyl-1,5-pentylene, 2,3-dimethyl-1,4-butylene, 1,7-heptylene, 2-methyl-1,6-hexylene, 3-methyl-1,6-hexylene, 2-ethyl-1,5-pentylene, 3-ethyl-1,5-pentylene, 2,3-dimethyl-1,5-pentylene, 2,4-dimethyl-1,5-pentylene, 1,8-octylene, 2-methyl-1,7-heptylene, 3-methyl-1,7-heptylene, 4-methyl-1,7-heptylene, 2-ethyl-1,6-hexylene, 3-ethyl-1,6-hexylene, 2,3-dimethyl-1,6-hexylene, 2,4-dimethyl-1,6-hexylene, 1,9-nonylene, 2-methyl-1,8-octylene, 3-methyl-1,8-octylene, 4-methyl-1,8-octylene, 2-ethyl-1,7-heptylene, 3-ethyl-1,7-heptylene, 1,10-decylene, 2-methyl-1,9-nonylene, 3-methyl-1,9-nonylene, 4-methyl-1,9-nonylene, 5-methyl-1,9-nonylene, 1,11-undecylene, 2-methyl-1,10-decylene, 3-methyl-1,10-decylene, 5-methyl-1,10-decylene, 1,12-dodecylene, 1,13-tridecylene, 1,14-tetradecylene, 1,15-pentadecylene, 1,16-hexadecylene, 1,17-heptadecylene, 1,18-octadecylene, 1,19-nonadecylene, 1,20-eicosylene and the like. Preferably, “C₁-C₃₀-alkylene” is unbranched or branched C₄-C₂₀-alkylene groups, particularly preferably unbranched or branched C₆-C₁₈-alkylene groups, in particular unbranched C₉-C₁₆-alkylene groups.

The expression “C₁-C₃₀-alkylene” includes in its definition also the expressions “C₄-C₃₀-alkylene”, “C₁₂-C₁₈-alkylene”, “C₆-C₁₅-alkylene”, “C₁₂-C₁₆-alkylene”, and “C₉-C₁₃-alkylene”.

In the context of the present invention, the expression “C₂-C₁₀-alkylene” refers to divalent hydrocarbon radicals having 2 to 10 carbon atoms. The divalent hydrocarbon radicals can be unbranched or branched. These include, for example, 1,2-ethylene, 1,2-propylene, 1,3-propylene, 1,2-butylene, 1,3-butylene, 1,4-butylene, 2-methyl-1,3-propylene, 1,1-dimethyl-1,2-ethylene, 1,5-pentylene, 1-methyl-1,4-butylene, 2-methyl-1,4-butylene, 1-ethyl-1,3-propylene, 2-ethyl-1,3-propylene, 1,1-dimethyl-1,3-propylene, 1,2-dimethyl-1,3-propylene, 2,2-dimethyl-1,3-propylene, 1,6-hexylene, 1-methyl-1,5-pentylene, 2-methyl-1,5-pentylene, 3-methyl-1,5-pentylene, 1-ethyl-1,4-butylene, 2-ethyl-1,4-butylene, 2-propyl-1,3-propylene, 1,1-dimethyl-1,4-butylene, 1,2-dimethyl-1,4-butylene, 2,2-dimethyl-1,4-butylene, 2,3-dimethyl-1,4-butylene, 1,7-heptylene, 2-methyl-1,6-hexylene, 3-methyl-1,6-hexylene, 2-ethyl-1,5-pentylene, 3-ethyl-1,5-pentylene, 2,3-dimethyl-1,5-pentylene, 2,4-dimethyl-1,5-pentylene, 1,8-octylene, 2-methyl-1,7-heptylene, 3-methyl-1,7-heptylene, 4-methyl-1,7-heptylene, 2-ethyl-1,6-hexylene, 3-ethyl-1,6-hexylene, 2,3-dimethyl-1,6-hexylene, 2,4-dimethyl-1,6-hexylene, 1,9-nonylene, 2-methyl-1,8-octylene, 3-methyl-1,8-octylene, 4-methyl-1,8-octylene, 2-ethyl-1,7-heptylene, 3-ethyl-1,7-heptylene, 1,10-decylene, 2-methyl-1,9-nonylene, 3-methyl-1,9-nonylene, 4-methyl-1,9-nonylene, 5-methyl-1,9-nonylene, and the like. Preferably, “C₂-C₁₀-alkylene” is unbranched C₂-C₆-alkylene groups or branched C₃-C₆-alkylene groups, particularly preferably unbranched C₂-C₄-alkylene groups or unbranched C₃-C₄-alkylene groups, in particular unbranched C₂-C₄-alkylene groups.

In the context of the present invention, “C₂-C₃₀-alkenylene” is divalent hydrocarbon radicals having 2 to 30 carbon atoms which may be unbranched or branched, where the main chain has 1, 2, or 3 double bonds. Preferably, the “C₂-C₃₀-alkenylene” is unbranched or branched C₆-C₁₈-alkenylene groups having 1, 2 or 3 double bonds, particularly preferably unbranched C₆-C₁₈-alkenylene groups having 1, 2 or 3 double bonds. These include, for example, 1-, 2-, 3-hexenylene, 1-, 2-, 3-heptenylene, 1-, 2-, 3-octenylene, 1-, 2-, 3-nonenylene, 1-, 2-, 3-, 4-, 5-decenylene, 1-, 2-, 3-, 4-, 5-undecenylene, 2-, 3-, 4-, 5-, 6-dodecenylene, 2,4-dodecadienylene, 2,5-dodecadienylene, 2,6-dodecadienylene, 3-, 4-, 5-, 6-tridecenylene, 2,5-tridecadienylene, 4,7-tridecadienylene, 5,8-tridecadienylene, 4-, 5-, 6-, 7-tetradecenylene, 2,5-tetradecadienylene, 4,7-tetradecadienylene, 5,8-tetradecadienylene, 4-, 5-, 6-, 7-pentadecenylene, 2,5-pentadecadienylene, 4,7-pentadecadienylene, 5,8-pentadecadienylene, 1,4,7-pentadecatrienylene, 4,7,11-pentadecatrienylene, 4,6,8-pentadecatrienylene, 4-, 5-, 6-, 7-, 8-hexadecenylene, 2,5-hexadecadienylene, 4,7-hexadecadienylene, 5,8-hexa-decadienylene, 2,5,8-hexadecatrienylene, 4,8,11-hexadecatrienylene, 5,7,9-hexadecatrienylene, 5-, 6-, 7-, 8-heptadecenylene, 2,5-heptadecadienylene, 4,7-heptadecadienylene, 5,8-heptadecadienylene, 5-, 6-, 7-, 8-, 9-octadecenylene, 2,5-octadecadienylene, 4,7-octadecadienylene, 5,8-octadecadienylene and the like. In particular, “C₂-C₃₀-alkenylene” is unbranched C₈-C₁₈-alkenylene groups with one or two double bonds, in particular unbranched C₉-C₁₆-alkenylene groups with one double bond.

The expression “C₂-C₃₀-alkenylene” includes in its definition also the expressions “C₄-C₃₀-alkenylene”, “C₁₂-C₁₈-alkenylene”, “C₆-C₁₅-alkenylene”, “C₁₂-C₁₆-alkenylene” and “C₉-C₁₃-alkenylene”.

The double bonds in the C₂-C₃₀-alkenylene groups can be present independently of one another in the E and also Z configuration or as a mixture of both configurations.

In the mono- or polybranched C₁-C₃₀-alkylene groups, C₂-C₁₈-alkylene groups and C₂-C₃₀-alkenylene groups, the carbon atom at the branching point or the carbon atoms at the respective branching points can have, independently of one another, an R or an S configuration or both configurations in equal or different proportions.

Preferably, the radical X¹ in the compounds of the general formula (I.a) has 11 to 21 ring carbon atoms and the radicals X² and Y in the compounds of the general formula (I.b) together have 9 to 19 ring carbon atoms.

Particularly preferably, the radical X¹ in the compounds of the general formula (I.a) has 12 to 18 ring carbon atoms, in particular 13 to 16 ring carbon atoms, and the radicals X² and Yin the compounds of the general formula (I.b) together have 10 to 17 ring carbon atoms, in particular 11 to 15 ring carbon atoms.

In a preferred embodiment of the present invention, in the compounds of the general formulae (I.a), (I.b), (II.a), (II.b),
R¹ is hydrogen or methyl,
X¹ is an unbranched C₁₂-C₁₈-alkylene group or an unbranched C₁₂-C₁₈-alkenylene group comprising one or two double bonds,
X² is an unbranched C₆-C₁₅-alkylene group or an unbranched C₆-C₁₅-alkenylene group comprising one or two double bonds,
Y is an unbranched C₂-C₄-alkylene group or branched C₃-C₄-alkylene group and
R² is hydrogen or an unbranched C₁-C₆-alkyl group or branched C₃-C₆-alkyl group,

where the radicals X² and Y together have 10 to 17 directly bridging carbon atoms.

In a particularly preferred embodiment of the present invention, in the compounds of the general formulae (I.a), (I.b), (II.a), (II.b)
R¹ is hydrogen or methyl,
X¹ is an unbranched C₁₂-C₁₆-alkylene group or an unbranched C₁₂-C₁₆-alkenylene group comprising a double bond,
X² is an unbranched C₉-C₁₃-alkylene group or an unbranched C₉-C₁₃-alkenylene group comprising a double bond,
Y is an unbranched C₂-C₄-alkylene group and
R² is hydrogen or an unbranched C₁-C₄-alkyl group,

where the radicals X² and Y together have 11 to 15 directly bridging carbon atoms.

In the context of the present invention, the expression “directly bridging carbon atoms” refers to the carbon atoms which link the terminal bonds in the shortest way.

Step a)

The at least one compound (II.a) provided in step a) of the process according to the invention or the at least one compound (II.b) can either be present in pure form or as an industrially available mixture which comprises at least one of the compounds (II.a) or (II.b). The content of compounds (II.a) or (II.b) is generally more than 50% by weight, preferably more than 60% by weight, in particular more than 70% by weight, based on the total weight of the industrially available mixture.

The compounds (II.a) or (II.b) can either be acquired commercially, synthesized or be produced by means of a biocatalytic process, for example via a fermentative or enzymatic process.

In a preferred embodiment of step a) of the process according to the invention, the compounds (II.a) or (II.b) are prepared from C₆-C₂₂-carboxylic acids, the provision of the compounds (II.a) and (II.b) comprising the following steps:
a.1) provision of a C₆-C₂₂-carboxylic acid,
a.2) conversion of the C₆-C₂₂-carboxylic acid provided in step a.1) to omega- and/or (omega-1)-hydroxylated or omega-carboxylated C₆-C₂₂-carboxylic acids,
a.3) optionally the oxidation of the omega-hydroxylated C₆-C₂₂-carboxylic acids obtained in step a.2) to the corresponding omega-carboxylated C₆-C₂₂-carboxylic acids,
a.4) optionally the esterification of the omega- and/or (omega-1)-hydroxylated C₆-C₂₂-carboxylic acids from step a.2) or of the carboxylated C₆-C₂₂-carboxylic acids from steps a.2) or a.3) with unbranched or branched C₁-C₆-alkanols.

The C₆-C₂₂-carboxylic acids used in step a.2) are generally commercially available and can be obtained in large amounts from readily accessible natural sources.

As a rule, the conversion of the C₆-C₂₂-carboxylic acids in step a.2) is carried out biocatalytically. The biocatalytic conversion in step a.2) can take place in different ways, such as, for example, by using catalytic amounts of a suitable enzyme or via a fermentative process. Preferably, the biocatalytic conversion in step a.2) takes place via a fermentative process.

For the fermentative conversion of the C₆-C₂₂-carboxylic acids to the corresponding omega- and/or (omega-1)-hydroxylated or omega-carboxylated C₆-C₂₂-carboxylic acids, a large number of suitable microorganisms are available to the person skilled in the art. For example, it is possible to use various yeast strains which belong to the genus Candida, such as Candida apicola, Candida albicans, Candida bombicola, Candida magnoliae or Candida tropicanea, for this purpose.

Preference is given in the process according to the invention for the fermentative conversion to using the yeast strains Candida bombicola, Candida magnoliae and Candida tropicanea.

Suitable fermentative processes are described in detail for example in the publications Spencer et al., Canadian Journal of Chemistry, 1961, vol. 39, pp. 846.

In the case of fermentative conversion using yeast strains, the omega- and/or (omega-1)-hydroxylated carboxylic acids or the (alpha, omega)-dicarboxylic acids are obtained in sophorose-bonded form, i.e. as so-called sophorolipids. The sophorolipids, which are usually present as aqueous suspension, are generally separated off by means of suitable separation processes, for example by extraction with organic solvents, from the remaining fermentative water-soluble residues. Subsequently, the hydroxylated or carboxylated carboxylic acids are cleaved off from the sophorose by acid hydrolysis.

Of suitability for the extraction of the sophorolipids are generally all organic solvents that are miscible with water only to a very low degree, if at all. Preferred organic solvents suitable for the extraction of these sophorolipids are selected, for example, from aliphatic or alicyclic hydrocarbons, such as pentane, hexane, heptane, ligroin, petrolether or cyclohexane, halogenated aliphatic or alicyclic hydrocarbons, such as dichloromethane, trichloromethane, tetrachloromethane, or dichloroethane, aromatic hydrocarbons, such as benzene, toluene or xylene, halogenated aromatic hydrocarbons, such as chlorobenzene or dichlorobenzene, ethers, such as methyl tert-butyl ether, diethyl ether, dibutyl ether, tetrahydrofuran, 1,4-dioxane, 1,2-dimethoxyethane, carboxylic acid esters, such as acetic acid methyl ester or acetic acid ethyl ester, and the like, as well as mixtures of said solvents.

In the case of fermentative hydroxylation, the C₆-C₂₂-carboxylic acids used are hydroxylated at the omega or omega-1 position. The hydroxylation can proceed selectively or unselectively, depending on which yeast strain and what conditions have been selected for the fermentation. Consequently, during fermentative hydroxylation, a mixture of omega- and (omega-1)-hydroxylated carboxylic acids are generally obtained which can be present in equal or different proportions. Mostly, the fermentative hydroxylation proceeds regioselectively, with one of the hydroxylation products being formed in excess.

Of suitability for cleaving off the hydroxylated or carbocyclized fatty acids from the sophorose are generally the processes known to the person skilled in the art for the acidic hydrolysis of ester and/or ether groups, as described for example in CH 430679 or DE 2834117. Preferably, the hydrolysis of the sophorolipids, i.e. the cleaving off of the hydroxylated or carbocyclized fatty acids from the sophorose, is carried out under acidic conditions, using mineral acids or organic sulfonic acids. For the hydrolysis of the sophorolipids, particular preference is given to using mineral acids, in particular sulfuric acid.

If desired, the omega-hydroxylated C₆-C₂₂-carboxylic acids obtained in step a.2) can be oxidized to the corresponding (alpha-omega)-dicarboxylic acids (step a.3)). Of suitability for this purpose are the customary processes known to the person skilled in the art for the oxidation of primary alcohols to carboxylic acids.

The omega- and/or (omega-1)-hydroxylated or omega-carboxylated C₆-C₂₂-carboxylic acids obtained in steps a.2) and a.3) are optionally converted in the presence of unbranched or branched C₁-C₆-alkanols, preferably in the presence of unbranched or branched C₁-C₄-alkanols, particularly preferably in the presence of unbranched C₁-C₄-alkanols, in particular in the presence of methanol or ethanol, to the corresponding carboxylic acid or dicarboxylic acid esters (step a.4)).

Esterification catalysts that can be used are the catalysts customary for this purpose, e.g. mineral acids, such as sulfuric acid and phosphoric acid; organic sulfonic acids, such as methanesulfonic acid and p-toluenesulfonic acid; amphoteric catalysts, in particular titanium, tin(IV) or zirconium compounds, such as tetraalkoxytitaniums, e.g. tetrabutoxytitanium, and tin(IV) oxide. The water which is formed during the reaction can be removed by customary measures, e.g. distillatively. As esterification catalysts, preference is given to using mineral acids or organic sulfonic acids, particularly preferably mineral acids, in particular sulfuric acid.

The esterification catalyst is used in an effective amount, which is usually in the range from 0.05 to 10% by weight, preferably 0.1 to 5% by weight, based on the acid component. Further detailed descriptions of suitable esterification processes can be found, for example, in U.S. Pat. No. 6,310,235, U.S. Pat. No. 5,324,853, DE-A 2612355 or DE-A 1945359.

The esterification (step a.4)) can take place during or after the hydrolysis of the sophorolipids carried out in step a.2). Preferably, the esterification is carried out during the hydrolysis, in which case the same mineral acid, in particular sulfuric acid, is used for the hydrolysis as well as for the esterification.

The at least one compound of the general formulae (II.a) or (II.b) obtained in steps a.2), a.3) or a.4) can, following work-up, be subjected to a further purification, for example a distillative purification, or be further used directly.

In a particularly preferred embodiment of step a) of the process according to the invention, C₆-C₂₂-carboxylic acids are converted fermentatively into the corresponding sophorose-bonded omega- and/or (omega-1)-hydroxylated or omega-carboxylated C₆-C₂₂-carboxylic acids, then cleaved off from the sophorose with the addition of a mineral acid in the presence of methanol or ethanol, with the simultaneous formation of the methyl and/or ethyl ester, and the crude esters obtained in this way are subjected to distillative purification or further used directly depending on the degree of purity.

The enzymatic or fermentative (omega-1)-hydroxylation can proceed enantioselectively. For this reason, the compounds of the general formula (II.a) prepared by means of enzymatic or fermentative (omega-1)-hydroxylation can be present as pure R or S isomers or as RIS isomer mixtures in which one of the enantiomers is present in excess.

Compounds of the general formula (II.a) or (II.b) which are present as pure R or S isomers or as RIS isomer mixtures and optionally additionally as pure E or Z isomers or E/Z isomer mixtures are equally suitable as feed materials for the process according to the invention.

Step b):

Step b) of the process according to the invention includes two variants. Variant b.1) of the process according to the invention relates to the conversion of at least one compound of the general formula (II.a) to a reaction mixture which comprises at least one macrocyclic compound of the general formula (I.a). Variant b.2) relates to the conversion of at least one compound of the general formula (II.b) and additionally at least one diol HO—Y—OH, to a reaction mixture which comprises at least one macrocyclic compound of the general formula (I.b).

In both variants, a product stream enriched in the macrocyclic compounds of the general formula (I.a) or (I.b) is removed by distillation from the reaction mixture.

The catalyst used in b.1) and b.2), the at least one poyether compound (PE), the reaction conditions, and the reaction procedure are identical in both variants b.1) and b.2). Consequently, variants b.1) and b.2) differ only in the type of feed materials used and the cyclization products obtained.

The fraction of starting material provided in step a) in the reaction mixture of steps b.1) or b.2) is generally 5 to 60% by weight, in particular 10 to 50% by weight, based on the total weight of the reaction mixture at the start of the conversion.

In the diol HO—Y—OH, according to the invention, Y is an unbranched or branched C₂-C₁₀-alkylene group, preferably an unbranched C₂-C₆-alkylene group or branched C₃-C₆ alkylene group, especially C₂-C₄-alkylene groups.

Removal from the system:

A product stream enriched in the macrocyclic compounds of the general formulae (I.a) or (I.b) is removed by distillation from the reaction mixture obtained during the conversion in steps b.1) and b.2). Consequently, the reaction mixture is separated into a product fraction enriched in the macrocyclic compounds of the general formula (I.a) or (I.b) and a bottom product enriched in the polyether compound (PE) and the catalyst.

Of suitability for the distillative removal of the macrocyclic compounds of the general formula (I.a) or (I.b) are generally all devices for the distillative separation of reaction mixtures which comprise liquid components. Suitable devices comprise distillation columns, such as tray columns, which may be equipped with bubble-cap trays, sieve plates, sieve trays, random packings or arranged packings, or spinning band column evaporators, such as thin-film evaporators, falling-film evaporators, forced-circulation evaporators, Sambay evaporators, etc. and combinations thereof. For the distillative removal of the macrocyclic compounds, particular preference is given to using distillation columns and/or spinning band columns, especially spinning band columns.

During the distillative removal of the product stream enriched in the macrocyclic compounds of the general formula (I.a) or (I.b), a vapor is firstly stripped off from the reaction mixture obtained in steps b.1) and b.2), and this is then at least partially condensed. All suitable condensers can be used for the condensation or partial condensation of the vapor. This may be cooled using any desired cooling media. Condensers with air cooling and/or water cooling are preferred, with air cooling being particularly preferred.

In a preferred embodiment of the process according to the invention, at least one solvent (S) different from the polyether compound (PE) is added as entrainer for the purposes of the distillative removal of the product stream enriched in the compounds (I.a) or (I.b) to the reaction mixture obtained in step b.1) or b.2), and/or an inert gas stream is introduced into the reaction mixture.

Solvent (S)

The at least one solvent (S) different from the polyether compound (PE) optionally added to the reaction mixture obtained in step b.1) or b.2) is intended to increase the rate of the distillative removal of the cyclization products (I.a) or (I.b) formed during the conversion. The solvent (S) thus fulfills the function of an entrainer.

In the context of the present invention, the expression “entrainer” is understood as meaning an organic compound, in particular an organic solvent, which converts at least partially to the gas phase together with the compounds (I.a) and/or (I.b).

Suitable solvents (S) are generally all solvents whose boiling point at 1013 mbar is in the range 95°-300° C. and which convert at least partially into the gas phase together with the compounds (I.a) and/or (I.b), but are immiscible or only slightly miscible with the compounds (I.a) and (I.b).

Preferably, the at least one solvent (S) is selected from C₂-C₁₅-alkanols, glycerol, pentaerythritol, C₂-C₄-alkylene glycols and their mono- and di-(C₁-C₄-alkyl) ethers, polyalkylene glycols different from the compounds PE and their mono- and dialkyl ethers which have a number-average molecular weight of less than 200 g/mol, aromatic hydrocarbons, and mixtures of the aforementioned solvents.

The at least one solvent (S) is particularly preferably glycerol, ethylene glycol, propylene glycol or polyalkylene glycols different from the compounds PE and their mono- and dialkyl ethers which have a number-average molecular weight of less than 200 g/mol. In particular, the at least one solvent (S) is glycerol and ethylene glycol.

Preferably, the at least one solvent (S) is metered into the reaction over a prolonged period.

Depending on the reaction procedure, the addition of the at least one solvent (S) takes place at the start of the reaction or at a later time in the course of the reaction.

Preferably, the at least one solvent (S) is metered in continuously during the entire course of the distillative removal of the cyclization products (I.a) or (I.b).

The amount of added solvents (S) is governed by the total amount of the compounds (II.a) or (II.b) used and the time required for separating off the cyclization products (I.a) or (I.b). In this connection, it has proven to be advantageous if the amount of added solvents (S) is in the range from 0.02 to 50 g/(g_{(starting material)}*h) (gram of S per gram of starting material and hour). Preferably, the amount of added solvents (S) is in the range from 0.03 to 25 g/(g_{(starting material)}*h), particularly preferably in the range from 0.05 to 10 g/(g_{(starting material)}*h), in particular in a range from 0.1 to 5 g/(g_{(starting material)}*h).

Inert gas stream:

Alternatively or additionally to the addition of the at least one solvent (S) to the reaction in steps b.1) or b.2), the distillative removal of the product stream enriched in the compounds (I.a) or (I.b) can take place with the introduction of a gas that is inert under the reaction conditions into the reaction mixture.

An inert gas is understood as meaning a gas which does not enter into any reactions with the starting materials involved in the reactions, reagents, solvents or the products that form under the stated process conditions. Suitable inert gases are e.g. nitrogen, helium, argon, etc. Preference is given to using nitrogen as inert gas.

For this, the inert gas can be passed into the gas space of the reaction zone or into the liquid reaction mixture. Preferably, the inert gas is introduced into the reaction zone in such a way that a large exchange area is created between the liquid reaction mixture and the inert gas. As also in the case of the addition of the at least one solvent (S) to the reaction mixture, the introduction of the inert gas brings about a stripping effect and facilitates the removal of the monomeric cyclization products from the reaction mixture.

Preferably, the inert gas is introduced into the boiling reaction mixture below the surface of the liquid such that it bubbles through the reaction mixture. The pressure of the inert gas must be sufficiently high to overcome the hydrostatic pressure of the reaction mixture above the inert gas feed point. E.g. the inert gas can be introduced 20 to 50 cm below the surface of the liquid of the reaction mixture.

The inert gas can be fed in via any desired suitable devices. These include e.g. gas-dispersion lances or nozzles. The nozzles can be provided on or in the vicinity of the reactor floor. The nozzles can be configured for this purpose as openings of a hollow chamber surrounding the reactor. Alternatively, immersion nozzles with suitable feed lines can be used. Several nozzles can be arranged e.g. in the form of a ring. The nozzles can point upwards or downwards. The nozzles preferably point sloping downwards.

The reaction mixture is preferably mixed thoroughly in order to bring about an exchange of reaction mixture in the reactor zone below the feed point of the inert gas with reaction mixture in the reactor zone above the feed point of the inert gas. Of suitability for the mixing are, for example, stirrers or circulation pump. In a specific variant, a so-called gas-dispersion stirrer is used to introduce the inert gas and mix the reaction mixture.

Reaction conditions steps b.1) or b.2):

The reaction in steps b.1) or b.2) proceeds in principle in two phases. In a first phase of the reaction, the oligomerization or polymerization phase, the feed materials oligomerize predominantly to linear polyesters of different chain length, where the macrocyclic compounds of the general formula (I.a) or (I.b) are formed only to a slight extent, if at all. In a second phase of the reaction, the oligomer esters are depolymerized and cyclized. For this, the monomeric cyclization product that is formed in the equilibrium is removed distillatively from the reaction mixture as the lowest-boiling component.

In a preferred embodiment of the process according to the invention, the distillative elimination of the product stream enriched in the compounds (I.a) or (I.b) takes place essentially after the reaction in step b.1) or b.2). The expression “essentially after the reaction” means in this connection that the distillative removal of the product stream enriched in the compounds (I.a) or (I.b) is started as soon as more than 70%, for example 80, 90 or 95%, of the feed materials of the general formulae (II.a) or (II.b) have been converted.

The phase of the depolymerization or cyclization generally starts with the removal of the monomeric cyclic product which is formed in the equilibrium from the reaction mixture, i.e. as soon as the distillative removal of the monomeric cyclization product is started. Since the oligomerization generally proceeds relatively rapidly, the distillative removal of the cyclization products (I.a) or (I.b) can take place just a few minutes after the start of the reaction in step b.1) or b.2), for example 5, 10 or 20 minutes after the start of the conversion, provided the reaction temperature and the pressure are already within the range required for this purpose.

The distillative removal of the product stream enriched in the compounds (I.a) or (I.b) takes place particularly preferably after the conversion in step b.1) or b.2).

The conversion in steps b.1) or b.2) and the distillative removal of the product stream enriched in the macrocyclic compounds of the general formula (I.a) or (I.b) are generally carried out at a temperature in the range from 150 to 350° C., preferably at a temperature in the range from 180 to 320° C. and in particular at a temperature in the range from 200 to 300° C.

The conversion in steps b.1) or b.2) can generally take place at ambient pressure or reduced pressure. Preferably, the reaction in steps b.1) or b.2) as well as the distillative removal of the product stream enriched in the macrocyclic compounds of the general formula (I.a) or (I.b) takes place at reduced pressure.

Polyether compound (PE)

Suitable as polyether compound (PE) are generally polyetherols with a number-average molecular weight of at least 200 g/mol, where their alcohol functions can be etherified with at least one (C₁-C₃₀)-alcohol and/or esterified with at least one (C₂-C₃₀)-carboxylic acid. The alcohol functions may likewise be present as free —OH groups.

They are preferably polyetherols wherein the alcohol functions may be etherified with a (C₁-C₁₀)-alcohol or esterified with a (C₂-C₁₀)-carboxylic acid.

Suitable polyetherols, and the specified ether and ester derivatives thereof, can be linear or branched, preferably linear. Suitable polyetherols generally have a number-average molecular weight in the range from about 200 to 20 000, preferably 250 to 5000, particularly preferably 300 to 3000.

Suitable polyetherols are, for example, nonionic polymers which have alkylene oxide repeat units. Preferably, the fraction of alkylene oxide repeat units is at least 30% by weight, based on the total weight of the compound. Suitable polyetherols are polyalkylene glycols, such as polyethylene glycols, polypropylene glycols, polytetrahydrofurans and alkylene oxide copolymers. Suitable alkylene oxides for the preparation of alkylene oxide copolymers are e.g. ethylene oxide, propylene oxide, epichlorohydrin, 1,2- and 2,3-butylene oxide. Of suitability are, for example, copolymers of ethylene oxide and propylene oxide, copolymers of ethylene oxide and butylene oxide, and copolymers of ethylene oxide, propylene oxide and at least one butylene oxide. The alkylene oxide copolymers can comprise the alkylene oxide units in polymerized-in form in statistical distribution or in the form of blocks. Preferably, the fraction of repeat units derived from ethylene oxide in the ethylene oxide/propylene oxide copolymers is 40 to 99% by weight. Of particular suitability as polyether compound (PE) are ethylene oxide homopolymers and ethylene oxide/propylene oxide copolymers.

Also suitable as polyether compound (PE) are ether and/or ester derivatives of the polyetherols described above which are derived from low molecular weight C₁-C₆-alcohols or from C₇-C₃₀-fatty alcohols and/or from low molecular weight C₂-C₆-carboxylic acids or from C₇-C₃₀-fatty acids.

These include, for example, polyalkylene glycol monoalcohol ethers, polyalkylene glycol dialkyl ethers, fatty alcohol polyoxyalkylene esters or polyalkylene glycol monofatty acid esters.

Of particular suitability are polyether compounds (PE) which distill off under the conditions chosen for the distillative removal only to a slight extent or only to a negligibly small extent together with the macrocyclic compounds of the general formula (I.a) or (I.b) and the solvent (S) optionally used for the distillative removal.

In a preferred embodiment of the process according to the invention, the polyether compound (PE) is selected from compounds which distill off only to a negligibly small extent, for example to less than 2% or to less than 1% or to less than 0.5%, based on the total amount of the polyether compound (PE) located in the reaction solution, together with the macrocyclic compounds of the general formula (I.a) or (I.b) and the solvent (S) optionally used for the distillative removal.

The polyether compounds used according to the invention preferably have a boiling point at 5 mbar of at least 280° C., particularly preferably of at least 300° C., in particular of at least 350° C.

In a further preferred embodiment of the process according to the invention, the polyether compound (PE) is selected from compounds of the general formula (III)

R³—O—[Z—O]ₙ—R³ (III)

in which
Z is selected independently of the others from ethylene, 1,2-propylene, 1,3-propylene, 1,2-butylene, 2,3-butylene and 1,4-butylene,
n is an integer from 3 to 250 and

either one radical R³ is hydrogen and the other radical R³ is C₁-C₃₀-alkyl, preferably C₁-C₁₀-alkyl, in particular C₁-C₆-alkyl, or

one radical R³ is hydrogen and the other radical R³ is —(C═O)—(C₁-C₃₀-alkyl), preferably —(C═O)—(C₁-C₁₀-alkyl), in particular —(C═O)—(C₁-C₆-alkyl), or

both radicals R³ are hydrogen or

both radicals R³ are —(C═O)—(C₁-C₃₀-alkyl), preferably —(C═O)—(C₁-C₁₀-alkyl), in particular —(C═O)—(C₁-C₆-alkyl).

In one embodiment, one radical R³ is hydrogen and the other radical R³ is C₁-C₃₀-alkyl, preferably C₁-C₁₀-alkyl, especially C₁-C₆-alkyl.

In another embodiment, one radical R³ is hydrogen and the other radical R³ is —(C═O)—(C₁-C₃₀-alkyl), preferably —(C═O)—(C₁-C₁₀-alkyl), especially —(C═O)—(C₁-C₆-alkyl).

In another embodiment, both radicals R³ are hydrogen.

In another embodiment, both radicals R³ are —(C═O)—(C₁-C₃₀-alkyl), preferably —(C═O)—(C₁-C₁₀-alkyl), especially —(C═O)—(C₁-C₆-alkyl).

Particularly preferably, either one radical R³ is hydrogen and the other radical R³ is C₁-C₁₀-alkyl, especially C₁-C₆-alkyl, or one radical R³ is hydrogen and the other radical R³ is —(C═O)—(C₁-C₁₀-alkyl), especially —(C═O)-(C₁-C₆-alkyl), or both radicals R³ are hydrogen.

As regards suitable and preferred compositions of the polyalkylene glycol repeat units

(Z) and the suitable and preferred average molecular weight of the compounds (III), that stated above is applicable. Accordingly, Z is preferably ethylene (homopolymers) or ethylene and 1,3-propylene (copolymers). Preferably, n is an integer from 4 to 100, particularly preferably from 5 to 50, in particular from 5 to 25.

As regards the terms “C₁-C₃₀-alkyl”, “C₁-C₁₀-alkyl” and “C₁-C₆-alkyl” used in the definition of R³, that stated above is likewise applicable.

Accordingly, the alkyl radicals in C₁-C₆-alkyl and —(C═O)—(C₁-C₆-alkyl) of R³ are for example unbranched C₁-C₆-alkyl groups or branched C₃-C₆-alkyl groups, preferably unbranched C₁-C₄-alkyl groups, and in particular methyl or ethyl.

In one embodiment preferred compounds of the formula (III) one R³ is hydrogen and the other R³ is hydrogen or C₁-C₁₀-alkyl or —(C═O)—(C₁-C₁₀-alkyl). In that case, preferably one R³ is hydrogen and the other R³ is hydrogen or C₁-C₆-alkyl or —(C═O)—(C₁-C₆-alkyl). In particular, both R³ are hydrogen.

Particularly preferred compounds of the general formula (III) are, for example, polyethylene glycols, polyethylene glycol monomethyl ether, polyethylene glycol monoethyl ether, polyethylene glycol monoacetate or polyethylene glycol diacetate, with an average molecular weight of 300 to 3000.

The fraction of the at least one polyether compound (PE) in the reaction mixture is generally 25 to 95% by weight, preferably 40 to 90% by weight, in particular 50 to 85% by weight, based on the total weight of the reaction mixture at the start of the conversion.

Catalyst

The at least one catalyst used in the steps b.1) and b.2) is selected from metal alkoxides.

In a preferred embodiment of the process according to the invention, the at least one catalyst used in the steps b.1) and b.2) is selected from metal alkoxides which distill off only to a negligibly small extent, for example to less than 2% by weight or to less than 1% by weight or to less than 0.5% by weight, based on the total amount of the at least one metal alkoxide catalyst located in the reaction solution, together with the macrocyclic compounds of the general formula (I.a) or (I.b) and the solvent (S) optionally used for the distillative removal. In this preferred embodiment, the at least one metal alkoxide catalyst generally has a boiling point at 5 mbar of more than 250° C. Preferably, the at least one metal alkoxide catalyst has a boiling point at 5 mbar of more than 280° C., particularly preferably of more than 300° C., in particular of more than 350° C.

The at least one metal alkoxide catalyst used in the steps b.1) or b.2) is prepared by reacting at least one metal compound, selected from metal alkoxides, alkyl metal oxides, metal salts or metal alkoxides of the general formula M[O(C₁-C₄-alkyl)]ₘ, where m has the values 1, 2, 3 or 4, with at least one polyether compound (PE) selected from compounds of the general formula (III).

The metal of the metal compound used for producing the at least one metal alkoxide catalyst is selected from alkali metals, alkaline earth metals, transition metals of the 4th, 7th, 8th, 9th and 12th group, and also metals and/or semi-metals of the 13th, 14th and 15th group of the Periodic Table of the Elements.

Preferably, the metal of the metal compound used for producing the at least one metal alkoxide catalyst is selected from K, Na, Ca, Mg, Ti, Zr, Mn, Fe, Co, Zn, Cd, Al, Ge, Sn, Pb and Sb, in particular from K, Na, Ca, Mg, Ti and Zn.

For producing the at least one metal alkoxide catalyst, particular preference is given to using metal oxides, metal hydroxides or metal alkoxides M[O(C₁-C₄-alkyl)]ₘ, where the metal is selected from K, Na, Ca, Mg, Ti, and Zn. Particularly preferred metallic starting materials for producing the at least one metal alkoxide catalyst are, for example, potassium hydroxide (KOH), sodium methanolate (NaOMe), calcium oxide (CaO), magnesium oxide (MgO), zinc oxide (ZnO), titanium(IV) ethanolate (Ti(OEt)₄), titanium(IV) isopropanolate (Ti(OiPr)₄) or titanium(IV) butanolate (Ti(OBu)₄).

Preferred polyether compounds of the general formula (III) used for producing the at least one metal alkoxide catalyst are those as defined above.

Usually, the polyether compound used for producing the at least one metal alkoxide catalyst is used in an at least 1.5-fold molar excess, preferably in an at least 2-fold molar excess, for example in a 4-fold, 15-fold or 30-fold molar excess, based on the amount of metal oxide, metal hydroxide or metal alkoxide M[O(C₁-C₅-alkyl)]ₘ, used.

The at least one metal alkoxide catalyst is generally produced at a temperature of 50 to 250° C., preferably at a temperature of 80 to 220° C. and in particular at a temperature of 100 to 200° C.

The at least one metal alkoxide catalyst can be produced in the absence or in the presence of an inert gas, as defined above. Preferably, the at least one metal alkoxide catalyst is produced with the addition of an inert gas, the inert gas used preferably being nitrogen.

The at least one metal alkoxide catalyst can generally be produced at ambient pressure or reduced or increased pressure. Preferably, the production of the at least one metal alkoxide catalyst is carried out at ambient pressure or reduced pressure.

The low-boiling components that are formed during the production of the at least one metal alkoxide catalyst are optionally removed by distillation.

In this connection, the expression “low-boiling component” refers to organic compounds which are liberated during the reaction of the at least one metal compound and have a boiling point of less than 180° C. at 1013 mbar. The low-boiling components are, for example, water, a C₁-C₆-alcohol or other organic solvents.

Preference is given to using an inert gas stream for the distillative removal of the low-boiling components optionally formed during the production of the at least one metal alkoxide catalyst.

The production of the at least one metal alkoxide catalyst particularly preferably takes place at ambient pressure, where the low-boiling components that are optionally formed in the process are removed from the reaction mixture by distillation with the help of a stream of nitrogen.

For this purpose, the nitrogen is passed into the gas space of the reaction zone or into the liquid reaction mixture. Preferably, the inert gas is introduced to the reaction zone in such a way that a large exchange area is created between the liquid reaction mixture and the inert gas. The introduction of the inert gas brings about a stripping effect and facilitates the distillative removal of the low-boiling components from the reaction mixture.

The at least one metal alkoxide catalyst is preferably produced in situ. In this connection, the expression “in situ” means that the catalyst can also be produced during the reaction (steps b.1) or b.2)), but before the distillative removal of the monomeric cyclization product.

The at least one metal alkoxide catalyst, however, is particularly preferably produced before the reaction in steps b.1) or b.2).

In particular, the at least one metal alkoxide catalyst is produced in the absence of the feed materials (I.a), (I.b) and of the diol HO—Y—OH.

The amount of metal alkoxide catalyst used in steps b.1) or b.2) is 0.1 to 50 mol %, preferably 1 to 40 mol % and in particular 3 to 30 mol %, based on the total amount of the compounds (II.a) or (II.b) in the reaction mixture.

In a particularly preferred embodiment of the process according to the invention, the same polyether compound (PE) is used for producing the at least one metal alkoxide catalyst and for the reaction of the compounds (I.a) or (I.b) to give the corresponding cyclic products (II.a) or (II.b), the polyether compound (PE) being selected from compounds of the general formula (III). As regards preferred and particularly preferred polyethers of the general formula (III), as well as their fraction in the reaction mixture, that stated above is applicable.

The product stream enriched in the macrocyclic compounds of the general formula (I.a) or (I.b) and removed from the reaction mixture can comprise at least some of the solvent (S) and optionally additionally some of the polyether compound (PE).

In a further preferred embodiment of the process according to the invention, the distillatively removed product stream enriched in the compounds (I.a) or (I.b) comprises at least some of the solvent (S) and optionally additionally some of the polyether compound (PE). To separate off the solvent (S) and the optionally present polyether compound (PE), the product stream is subjected to a separation, giving a fraction enriched in the solvent (S) and optionally in the polyether compound (PE) and a product fraction which comprises predominantly macrocyclic compounds of the general formula (I.a) or (I.b).

Usually, the separation of the removed product stream into a fraction enriched in the solvent (S) and optionally in the polyether compound (PE) and a product fraction takes place via a process of self-separation (phase separation) if the solvent (S) and optionally the polyether compound (PE) is only slightly miscible, or completely immiscible, with the macrocyclic compounds of the general formula (I.a) or (I.b).

For this, the product stream is usually passed into a phase separator (decanter), where it disintegrates, as a result of mechanical settling, into two phases (a S phase which optionally comprises some of the polyether compound, and a product phase), which can be stripped off separately.

Otherwise, or in addition to this, the customary methods generally known to the person skilled in the art for separating liquid mixtures, such as distillation, liquid extraction or liquid chromatographic separation methods can be used for separating the removed product stream.

If the separation cannot be achieved, or can be achieved only incompletely, by the route of self-separation (phase separation), this can take place distillatively or also extractively. In the case of an extractive separation, this advantageously takes place using a solvent different from S which very readily dissolves the macrocyclic compounds of the general formula (I.a) or (I.b) but is only slightly miscible, or not miscible at all, with the solvent (S) and optionally with the polyether compound (PE).

Suitable solvents different from S are selected, for example, from aliphatic hydrocarbons, such as pentane, hexane, heptane, ligroin, petroleum ether, cyclopentane or cyclohexane, halogenated aliphatic hydrocarbons, such as dichloromethane, trichloromethane, tetrachloromethane, or 1,2-dichloroethane aromatic hydrocarbons, such as benzene, toluene, xylene, halogenated aromatic hydrocarbons, such as chlorobenzene, dichlorobenzenes, ethers, such as diethyl ethers, methyl tert-butyl ether, dibutyl ether, tetrahydrofuran or dioxane, and C₁-C₄-alkylnitriles, such as acetonitrile or propionitrile, and the like.

The product fraction obtained after the separation can, if required, be subjected to a further purification. Preferably, the further purification is a distillative separation.

Of suitability for the distillative separation of the product fraction are generally the devices specified in the statements relating to distillative removal. For the distillative separation of the product fraction, a fractionated distillation is preferably carried out using distillation columns or spinning band columns, in particular spinning band columns.

In a preferred embodiment of the process according to the invention, the fraction enriched in the solvent (S) and optionally in the polyether compound (FE) is returned again to the conversion in step b.1) or b.2).

“Returning to the conversion in step b.1) or b.2)” means that the solvent (S) and optionally the polyether compound (PE) is again passed back to the reaction zone of the conversion.

In the process according to the invention, the reaction zone can consist of a reactor or an arrangement of several reactors. Several reactors are preferably connected in series. The process according to the invention can be carried out discontinuously or continuously.

In a preferred embodiment of the process according to the invention, steps b.1) and b.2) and the distillative removal of the product stream enriched in the compounds (I.a) or (I.b) are carried out continuously.

The reactors may be any desired reactors which are suitable for carrying out chemical reactions in liquid phase.

Suitable reactors are non-back-mixed reactors, such as tubular reactors or dwell-time containers provided with internals, but preferably back-mixed reactors such as stirred-tank reactors, loop reactors, jet loop reactors or jet nozzle reactors. However, it is also possible to use combinations of successive back-mixed reactors and non-back-mixed reactors.

Optionally, several reactors can also be combined in a multistage apparatus. Such reactors are, for example, loop reactors with incorporated sieve trays, cascaded containers, tubular reactors with interim feed point or stirred columns.

Preference is given to using stirred-tank reactors. The stirred-tank reactors mostly consist of metallic materials, with stainless steel being preferred. The reaction batch is preferably mixed intensively with the help of a stirrer or a circulation pump.

In a preferred embodiment, the process according to the invention is carried out in a single stirred-tank reactor. In a further preferred embodiment, the process according to the invention is carried out in at least two stirred-tank reactors joined together in the form of a cascade. Specifically in the case of a continuous reaction procedure, it may be expedient for as complete as possible a conversion to join several reactors in the form of a cascade. The reaction mixture passes through the individual reactors in succession, the run-off from the first reactor being passed to the second reactor, the run-off from the second reactor being passed to the third reactor etc. The cascade can comprise e.g. 2 to 10 reactors, with 2, 3, 4 or 5 reactors being preferred.

If a cascade of several reactors is used for carrying out steps b.1), b.2), then all of the reactors in a cascade can be operated at the same temperature. However, it is generally preferred to steadily increase the temperature from the first to the last reactor of a cascade, with a reactor being operated at identical or higher temperature than the reactor positioned upstream in the flow direction of the reaction mixture. All of the reactors can expediently be operated at essentially identical pressure.

In the case of a continuous process procedure, streams of the starting materials and optionally of the solvent (S) are introduced into the reactor, or when using a reactor cascade preferably into the first reactor of the cascade, which comprises the catalyst and the polyether compound (PE). The residence time in the reactor or the individual reactors is determined here by the volume of the reactors and the quantity stream of the starting materials. A vapor comprising the monomeric cyclization product and at least some of the solvent (S) and optionally additionally some of the polyether compound (PE) is drawn off from the reactor or the individual reactors. After separating off the solvent (5) and optionally the polyether compound (PE) from the cyclization product, these are returned again to the reactor or the reactor cascade.

The vapor from the individual reactors of a cascade can be combined and condensed together. Optionally, it is possible in each case to combine several reactors of the cascade to give a subunit, in which case the subunits are then in each case coupled to a condenser. There is also furthermore the option of coupling each reactor of the cascade to a condenser.

The solvent (S) to be returned and the polyether compound (PE) optionally to be returned can be passed to any desired reactor of a cascade or be divided between several reactors of the cascade. However, it is preferred to pass the solvent (S) to be returned and the polyether compound (PE) optionally to be returned not to the last reactor of the cascade. Preferably, the solvent (S) to be returned and the polyether compound (PE) optionally to be returned are passed exclusively or predominantly to the first reactor of the cascade.

The invention further provides macrocyclic lactones of the general formula (I.a) which are selected from (10Z,18S)-18-methyl-1-oxacyclooctadec-10-en-2-one, (10Z,18R)-18-methyl-1-oxacyclooctadec-10-en-2-one, (10E,18S)-18-methyl-1-oxacyclooctadec-10-en-2-one and (10E,18R)-18-methyl-1-oxacyclooctadec-10-en-2-one.

The present invention relates both to the aforementioned isomeric compounds in their pure form as well as mixtures thereof.

The term “18-methyl-1-oxacyclooctadec-10-en-2-one” used below thus refers both to the individual aforementioned isomers of 18-methyl-1-oxacyclooctadec-10-en-2-one as well as to mixtures thereof.

In the mixtures, the compounds can be present in equal proportions or one of the compounds can be present in excess. Preferably, one of the compounds is present to at least 60% by weight, in particular to at least 80% by weight and specifically to at least 90% by weight, based on the total amount of the isomer compounds present in the mixture.

The aforementioned isomers of 18-methyl-1-oxacyclooctadec-10-en-2-one can all be prepared with the help of the process according to the invention and have advantageous sensory properties, in particular a pleasant odor. Specifically, the isomers of 18-methyl-1-oxacyclooctadec-10-en-2-one have a concise musk-like odor.

For this reason, the invention likewise relates to the use of at least one compound which is selected from (10Z,18S)-18-methyl-1-oxacyclooctadec-10-en-2-one, (10Z,18R)-18-methyl-1-oxacyclooctadec-10-en-2-one, (10E,18S)-18-methyl-1-oxacyclooctadec-10-en-2-one and (10E,18R)-18-methyl-1-oxacyclooctadec-10-en-2-one as fragrance and/or flavoring. The compounds used for this purpose have a purity of at least 80%, in particular of at least 90%, for example of 95% or 97%.

Intense odor impressions are to be understood as meaning those properties of aroma chemicals which permit a striking perception even in very low gas space concentrations. The intensity can be determined via a threshold value determination. A threshold value is the concentration of a substance in the relevant gas space at which an odor impression can just still be perceived by a representative test panel, although it no longer has to be defined. A substance class which probably belongs to the most odor-intensive known substance classes, i.e. has very low odor threshold values, are thiols, whose threshold value is often in the ppb/m³ range. The aim of searching for novel aroma chemicals is to find substances with the lowest possible odor threshold value in order to permit the lowest possible use concentration. The closer one comes to this target, the more one speaks of “intense” odor substances or aroma chemicals.

“Advantageous sensory properties” or “pleasant odor” are hedonistic expressions which describe the niceness and conciseness of an odor impression conveyed by an aroma chemical.

“Niceness” and “conciseness” are terms which are familiar to the person skilled in the art, a perfumer. Niceness generally refers to a spontaneously brought about, positively perceived, pleasant sensory impression. However, “nice” does not have to be synonymous with “sweet”. “Nice” can also be the odor of musk or sandalwood. “Conciseness” generally refers to a spontaneously brought about sensory impression which—for the same test panel—brings about a reproducibly identical reminder of something specific.

For example, a substance can have an odor which is spontaneously reminiscent of that of an “apple”: the odor would then be concisely of “apples”. If this apple odor were very pleasant because the odor is reminiscent, for example, of a sweet, fully ripe apple, the odor would be termed “nice”. However, the odor of a typically tart apple can also be concise. If both reactions arise upon smelling the substance, in the example thus a nice and concise apple odor, then this substance has particularly advantageous sensory properties.

Furthermore, the present invention relates to the use of at least one compound which is selected from (10Z,18S)-18-methyl-1-oxacyclooctadec-10-en-2-one, (10Z,18R)-18-methyl-1-oxacyclooctadec-10-en-2-one, (10E,18S)-18-methyl-1-oxacyclooctadec-10-en-2-one and (10E,18R)-18-methyl-1-oxacyclooctadec-10-en-2-one, as constituent of a composition which typically comprises at least one aroma substance, i.e. fragrance and/or flavoring, as well as additionally a carrier material. Such compositions are selected, for example, from detergents, such as laundry care compositions, cleaners, cosmetic preparations, scent-containing hygiene articles, such as diapers, sanitary towels, armpit pads, paper towels, wet wipes, toilet paper, pocket tissues and the like, food and food supplements, such as chewing gums or vitamin products, scent dispensers, such as air fresheners, perfumes, pharmaceutical preparations and crop protection compositions.

In a preferred embodiment of the present invention, 18-methyl-1-oxacyclooctadec-10-en-2-one, as defined above, is used as a constituent in cosmetic preparations, scent-containing hygiene articles or perfumes.

For the formulation of these compositions, 18-methyl-1-oxacyclooctadec-10-en-2-one, as defined above, optionally together with one or more other aroma substances, is usually added to an existing preparation which comprises no aroma substances or one or more other aroma substances beforehand. Usually, these compositions additionally comprise a carrier material which can consist of a compound, a mixture of compounds or of different additives which have no or no noteworthy sensory properties. However, the carrier material can also be a compound or an additive which has the noteworthy sensory properties, or can be a mixture of compounds which comprises at least one aroma substance different from the isomers of 18-methyl-1-oxacyclooctadec-10-en-2-one and optionally at least one further compound which has no noteworthy sensory properties.

The carrier material can be a compound, a mixture of compounds or other additives which have the aforementioned properties. Suitable carrier materials comprise liquid or oil-like carrier materials as well as wax-like or solid carrier materials.

Suitable liquid or oil-like carrier materials are selected, for example, from water, alcohols, such as methanol or ethanol, aliphatic diols and polyols with a melting temperature below 20° C., such as ethylene glycol, glycerol, diglycerol, propylene glycol or dipropylene glycol, cyclic siloxanes, such as hexamethylcyclotrisiloxane or decamethylcyclopentasiloxane, vegetable oils, such as fractionated coconut oil or esters of fatty alcohols with melting temperatures below 20° C., such as tetradecyl acetate or tetradecyl lactate, and alkyl esters of fatty acids with melting temperatures below 20° C., such as isopropyl myristate.

Suitable wax-like or solid carrier materials are selected, for example, from fatty alcohols with melting temperatures below 20° C., such as myristyl alcohol, stearyl alcohol or cetyl alcohol, polyols with melting temperatures above 20° C., fatty acid esters with fatty alcohols which have a melting temperature of above 20° C., such as lanolin, beeswax, carnauba wax, candelilla wax or Japan wax, waxes produced from petroleum, such as hard paraffin, water-insoluble porous minerals, such as silica gel, silicates, for example talc, microporous crystalline aluminosilicates (zeolites), clay minerals, for example bentonite, or phosphates, for example sodium tripolyphosphate, paper, cardboard, wood, textile composite or nonwoven materials made of natural and/or synthetic fibers.

Suitable carrier materials are also selected, for example, from water-soluble polymers, such as polyacrylic acid esters or quaternized polyvinylpyrrolidones, or water-alcohol-soluble polymers, such as specific thermoplastic polyesters and polyamides. The polymeric carrier material can be present in various forms, e.g. in the form of a gel, a paste, solid paricles, such as microcapsules, or brittle coatings.

As a rule, the use amounts of 18-methyl-1-oxacyclooctadec-10-en-2-one in these compositions correspond to the customary standard commercial use amounts for additives in formulations. In order to be more precise, the use amount of 18-methyl-1-oxacyclooctadec-10-en-2-one is in the range from 0.001 to 50% by weight, in particular in the range from 0.01 to 20% by weight and specifically in the range from 0.1 to 10% by weight, based on the total weight of the composition.

Depending on their intended use, the compositions in which 18-methyl-1-oxacyclooctadec-10-en-2-one, as defined above, is used as odor-imparting constituent can comprise further auxiliaries and/or additives, such as, for example, detergents or mixtures of detergents, thickeners, such as polyethylene glycols with a number-average molecular weight of 400 to 20 000 Da, lubricants, binders or agglomerating agents, such as sodium silicates, dispersants, builder salts, water softeners, filling salts, pigments, colorants, optical brighteners, soil carriers and the like.

Furthermore, the present invention relates to a scent composition and/or a fragrance material comprising at least one isomer of 18-methyl-1-oxacyclooctadec-10-en-2-one, as defined above, and a carrier material.

The total concentration of 18-methyl-1-oxacyclooctadec-10-en-2-one in the scent composition according to the invention and/or the fragrance material according to the invention is not specifically limited. This can be adapted to the particular intended use within a wide range. As a rule, the customary standard commercial use amounts for scents are used. Usually, the total amount of 18-methyl-1-oxacyclooctadec-10-en-2-one in the scent composition according to the invention and/or the fragrance material according to the invention is in the range from 0.0001 to 20% by weight and in particular in the range from 0.001 to 10% by weight.

Typical fields of application of the scent compositions according to the invention and/or fragrance materials are detergents, textile care compositions, cleaners, preparations of scents for the human or animal body, for rooms such as kitchens, wet rooms, cars or lorries, for real or artificial plants, for clothing, for shoes and insoles, for items of furniture, for carpets, for room humidifiers, for air fresheners, for perfumes or for cosmetics such as ointments, creams, gels, shampoos, soaps or powders.

In particular, the scent compositions and/or fragrance materials according to the invention can be used in scent preparations for the human or animal body, for cosmetics such as ointments, creams, gels, shampoos, soaps or powders or in perfumes.

The invention also encompasses fragrance combinations which comprise 18-methyl-1-oxacyclooctadec-10-en-2-one, as defined above, as component A, and at least one further compound known as scent or aroma substance as component B, such as, for example, one or more of the following compounds B1 to B11:
B1: methyl dihydrojasmonate (e.g. hedione),
B2: 4,6,6,7,8,8-hexamethyl-1,3,4,6,7,8-hexahydrocyclopenta[g]benzopyran (e.g. Galaxolide™),
B3: 2-methyl-3-(4-tert-butylphenyl)propanal (Lysmeral™),
B4: 2-methyl-3-(4-isopropylphenyl)propanal (cyciamenaldehyde),
B5: 2,6-dimethyl-7-octen-2-ol (dihydromyrcenol),
B6: 3,7-dimethyl-1,6-octadien-3-ol (linalool),
B7: 3,7-dimethyl-trans-2,6-octadien-1-ol (geraniol),
B8: 2,3,8,8-tetramethyl-1,2,3,4,5,6,7,8-octahydro-2-naphthalenyl methyl ketone (Iso E Super™),
B9: alpha-hexylcinnamaldehyde,
B10: 3,7-dimethyl-6-octen-1-ol (citronellol),
B11: alpha, or beta-, or delta-damascone.

Suitable as formulations of fragrances are, for example, the formulations disclosed in JP 11-071312 A, paragraphs [0090] to [0092]. Likewise of suitability are also the formulations from JP 11-035969 A, paragraphs [0039] to [0043].

Furthermore, the present invention relates to a method for imparting or altering an odor or taste of a composition, in which 18-methyl-1-oxacyclooctadec-10-en-2-one is added to the composition in an amount which imparts an odor or taste to the composition or alters the odor or taste of the composition. The amounts of 18-methyl-1-oxacyclooctadec-10-en-2-one required for this depend on the nature and the intended use of the composition and can therefore vary widely. As a rule, the use amounts of 18-methyl-1-oxacyclooctadec-10-en-2-one, as defined above, are usually in the range from 0.0001 to 50% by weight, in particular in the range from 0.001 to 20% by weight, based on the total weight of the composition.

The invention is illustrated in more detail by reference to the examples described below. The examples here should not be understood as being limiting for the invention.

The following abbreviations are used in the examples below:
Eq is equivalents
EG is ethylene glycol
PEG is polyethylene glycol
Pluriol® E 600 S is polyethylene glycol with a number-average molecular weight of 600
Acetic ester is ethyl acetate
GC is gas chromatography
GC area % is the percentage fraction of the area of the substance peak based on the total area of the peak in a gas chromatogram (GC area percent).
RT is room temperature
Ti(OiPr)₄ is titanium(IV) isopropanolate

### EXAMPLES

### II) Preparation Examples

### Example II.1

### Cyclization of 15-hydroxypentadecanoic acid butyl ester to 15-penta-decanolide

At room temperature, 2.48 g of titanium(IV) isopropoxide (0.01 mol, 0.10 eq) are added to 80 g of Pluriol® 2000 (PEG 2000). The mixture is heated to 140 to 150° C. and the resulting isopropanol is stripped from the solution in approx. 3 h with nitrogen. Then, the mixture is cooled to 120° C. and 36.0 g (0.11 mol) of the 15-hydroxypentadecanoic acid butyl ester melted at 70° C. are added, and the mixture is evacuated to 5 mbar and heated to 250° C. over the course of approx. 20 min. At 250° C., the metered addition of ethylene glycol is started (approx. 25 ml/h), whereupon a mixture of pentadecanolide and ethylene glycol distills off. After approx. 6 h, the distillate is single-phase and, following phase separation, 25.5 g of pentadecanolide are obtained with a content of 96.8% by weight, which corresponds to a yield of 89.7%. A further 4.3% of product are obtained in the ethylene glycol phase.

### Example II.2

### Cyclization of 15-hydroxypentadecanoic acid methyl ester to 15-penta-decanolide

At room temperature, 2.84 g of titanium(IV) isopropoxide (0.01 mol, 0.1 eq) are added to 80 g of Pluriol® E 600 S. The mixture is heated to 140 to 145° C. and the resulting isopropanol is stripped from the solution in approx. 3 h with nitrogen. Then, the mixture is cooled to 120° C. and 28.7 g (0.1 mol) of the 15-hydroxypentadecanoic acid methyl ester melted at 70° C. are added, and the mixture is evacuated to 5 mbar and heated to 250° C. over the course of approx. 20 min. It can optionally also be cooled to room temperature and the addition of the hydroxyester take place at RT, the catalyst can moreover be stored. At 250° C., the metered addition of ethylene glycol is started (approx. 28 ml/h), whereupon a mixture of pentadecanolide and ethylene glycol distills off. After approx. 5 h, the distillate is single-phase and, following phase separation, 22.7 g of pentadecanolide with a content of 98.3% by weight are obtained, which corresponds to a yield of 92.9%. A further 2.4% of product are obtained in the ethylene glycol phase.

### Example II.3

### Cyclization of 15-hydroxypentadecanoic acid methyl ester to 15-pentadecanolide

At room temperature, 0,8 g of magnesium oxide (0.02 mol, 0.2 eq) are added to 80 g of Pluriol® E 600 S and, after heating to 120° C., 31.4 g (0.1 mol) of the 15-hydroxy-pentadecanoic acid butyl ester melted at 70° C. are added. Then, the mixture is evacuated to 5 mbar and heated to 250° C. over the course of approx, 20 min. At 250° C., the metered addition of ethylene glycol is started (approx. 20 ml/h), whereupon a mixture of pentadecanolide and ethylene glycol distills off. After approx. 10 h, the distillate is single-phase and, after phase separation, 21.1 g of pentadecanolide with a content of 97.9% by weight are obtained, which corresponds to a yield of 86.0%. A further 3.8% of product are obtained in the ethylene glycol phase.

### Examples II.4 to II.7 cyclization of 15-hydroxypentadecanoic acid methyl ester to 15-pentadecanolide using various catalysts

The preparation examples II.4 to II.7 were carried out analogously to examples 11.2 and II.3, further different metal salts being used for producing the catalyst.

The feed materials and reaction conditions used in examples II.1 to II.7 are summarized below in table 1:

TABLE 1
reaction conditions examples II.1 to II.7
Cyclization of 15-hydroxypentadecanoic acid methyl ester to 15-pentadecanolide

| | | | Reaction conditions | | | | |
|---|---|---|---|---|---|---|---|
| | | Amount of | T (upon | | | Reaction | Rate of S |
| | | starting | addition of | T | | time at | addition (EG) |
| | PE | material | starting material) | (depolymerization) | Pressure | T = 250° C. | [g/(g_{(starting material)} |
| Example | component | [% by weight] | [° C.] | [° C.] | [mbar] | [h] | *h)] |
| II.1 | PEG 2000 | 31 | 120 | 250 | 5 | 6 | 0.8 |
| II.2 | PEG E600S | 32 | RT | 250 | 5 | 6 | 0.6 |
| II.3 | PEG E600S | 28 | RT | 250 | 5 | 10 | 0.7 |
| II.4 | PEG E600S | 28 | RT | 250 | 5 | 6 | 0.8 |
| II.5 | PEG E600S | 29 | RT | 250 | 5 | 6 | 0.7 |
| II.6 | PEG E600S | 28 | RT | 250 | 5 | 6 | 0.9 |
| II.7 | PEG E600S | 28 | RT | 250 | 5 | 6 | 0.8 |

The catalysts used in preparation examples II.1 to II.7 and the yields of 15-penta-decanolide achieved are shown below in table 2.

TABLE 2
Yields examples II.1 to II.7
Cyclization of 15-hydroxypentadecanoic
acid methyl ester to 15-pentadecanolide

| | Catalyst | | | | |
|---|---|---|---|---|---|
| | | Amount with | | | |
| | | regard to | Yield of | | |
| | | starting | 15-pentadecanolide | | |
| Ex- | Metal salt | material | Distillate | In EG phase | Total |
| ample | used | [mol-%] | [%] | [%] | [%] |
| II.1 | Ti(OiPr)₄ | 10 | 92 | 4 | 97 |
| II.2 | Ti(OiPr)₄ | 10 | 94 | 5 | 99 |
| II.3 | MgO | 20 | 89 | 4 | 92 |
| II.4 | NaOMe | 10 | 85 | 5 | 90 |
| II.5 | ZnO | 20 | 91 | 3 | 94 |
| II.6 | KOH | 20 | 78 | 7 | 85 |
| II.7 | CaO | 20 | 71 | 7 | 78 |

III) Preparation of (omega-1)- and omega-hydroxy fatty acids are the cyclization thereof

### Example III.1a

### Isolation and Preparation of (Omega-1)- and Omega-Hydroxyoleic Acid Methyl Eester from Fermentatively Obtained Sophorolipids

160.8 g of an aqueous sophorolipid solution are extracted three times with in each case 400 ml of acetic ester at room temperature. The combined acetic ester phases are concentrated, giving a residue of 68,6 g. The residue is dissolved in 250 g of methanol and 6.9 g of concentrated sulfuric acid are added at RT. Then, the batch is heated at reflux for 10 h. The reaction solution is cooled and 13.8 g of potassium carbonate are added and the mixture is after-stirred at RT for 30 min. The suspension is filtered and the filtrate is concentrated by evaporation. The final weight is 81.6 g. The product is taken up in 400 ml of ethyl acetate and 400 ml of water and extracted. Following phase separation, the aqueous phase is extracted again with 400 ml of acetic ester. The ethyl acetate phases are combined and concentrated by evaporation, with a solid being obtained after cooling to room temperature. The final weight is 31.7 g. The mass yield is 19.7%, the content of hydroxyoleic acid methyl esters approx. 75.7% by weight. The ratio of (omega-1)- to omega-hydroxyoleic acid methyl ester is 6.4:1.

### Example III.1b

### Cyclization of a mixture of (omega-1)- and omega-hydroxyoleic acid methyl ester to (10Z)-18-methyl-1-oxacyclooctadec-10-en-2-one and (10Z)-1-oxacyclononadec-10-en-2-one

At room temperature, 2.84 g of titanium(IV) isopropoxide (M=284.22 g/mol, 0.01 mol) are added to 80 g of Pluriol® E 600 S. Then, the mixture is heated to 140 to 145° C. and the resulting isopropanol is stripped from the solution in approx. 3 h with nitrogen and the solution is cooled to RT. Over a period of less than 5 minutes, 31.3 g (approx. 75.7% by weight, 0.08 mol) of the mixture of (omega-1)-hydroxyoleic acid methyl ester and omega-hydroxyoleic acid methyl ester (omega-1:omega=6.4:1) isolated in the preceding step (example III.1.b) are added to the catalyst. The addition takes place here in less than 5 minutes. Then, the mixture is evacuated to 5 mbar and heated to 250° C. At 250° C., the metered addition of ethylene glycol (approx. 25-30 ml/h) is started and the product is thus stripped from the reaction mixture. After approx. 10 h, the reaction is complete and, after phase separation, 18.0 g of a mixture of (10Z)-18-methyl-1-oxacyclooctadec-10-en-2-one and (10Z)-1-oxacyclononadec-10-en-2-one with an isomer ratio of 7.3:1 and a content of 91% are calculated via the area fraction of the product peaks (both isomers) in the gas chromatogram (GC area %). This corresponds to a yield of 85% t.q. or 77% GC area %.

### Example III.2a

### Isolation and Preparation of (Omega-1)- and Omega-Hydroxypalmitic Acid Methyl Ester from Fermentatively Obtained Sophorolipids

985.0 g of the aqueous sophorolipid solution obtained from example III.2a are extracted three times with in each case 1000 ml of heptane at 70° C. and three times with in each case 1000 ml of acetic acid at room temperature. The combined acetic ester phases are concentrated by evaporation, giving a residue I of 573.9 g. The residue I is dissolved in 2870 g of methanol and 59.2 g of concentrated sulfuric acid are added at RT. Then, the mixture is heated at reflux for 10 h. The reaction solution is cooled, 108.4 g of potassium carbonate (1.3 eq based on sulfuric acid) are added and the mixture is after-stirred for 30 min at RT. The suspension is filtered and the filtrate is concentrated by evaporation. The final weight of the residue II is 630.7 g. This is taken up in 2600 ml of ethyl acetate and 1300 ml of water and extracted. After phase separation, the aqueous phase is extracted again with 2600 ml of acetic ester. The ethyl acetate phases are combined and concentrated by evaporation, with a solid being obtained after cooling to room temperature. The final weight is 308 g. The content of hydroxypalmitic acid methyl esters in the solid is approx. 45% by weight, which corresponds to a mass yield of hydroxypalmitic acid methyl esters of 31.3%. The ratio of (omega-1)- to omega-hydroxypalmitic acid methyl ester of the crude product is 1.1:1.

The mixture of the crude hydroxypalmitic acid methyl ester is fractionally distilled at 180° C. and 3 mbar. This gives approx. 81% by weight of the crude product used as distillation discharge. The average purity is approx. 67 GC % by weight or 78 GC area % (sum of both isomers), which corresponds to a mass yield of hydroxypalmitic acid methyl esters of 55%. The average isomer ratio of (omega-1)- to omega-hydroxypalmitic acid methyl ester after the distillation is 1.2:1.

### Example III.2b

### Cyclization of a mixture of omega- and omega-1-hydroxyhexadecanoic acid methyl ester to oxacycloheptadecan-2-one and 16-methyloxacyclohexadecan-2-one

At room temperature, 2.84 g of titanium(IV) isopropoxide (M=284.22 g/mol, 0.01 mol, 0.1 eq) are added to 80 g of Pluriol® E 600 S. Then, the mixture is heated to 140 to 145° C. and the resulting isopropanol is stripped from the solution in approx. 2 hr with nitrogen and the solution is cooled to RT. 40 g (approx. 71% by weight, 0.1 mol) of a melt (70° C.) of the mixture of (omega-1)-hydroxypalmitic acid methyl ester and omega-hydroxypalmitic acid methyl ester (omega-1:omega=1.2:1) isolated in the previous step (example III,2.b) are added at RT to the catalyst over a period of less than 5 minutes. Then, the mixture is evacuated to 5 mbar and heated to 250° C. over the course of approx. 20 min. As soon as 250° C. are reached, the metered addition of ethylene glycol is started (approx. 30 g/h). After approx. 6 h, the reaction is complete and, after phase separation, approx. 27.7 g of a mixture of 16-methyloxacyclo-hexadecan-2-one and oxacycloheptadecan-2-one with a purity of 91.1 GC area % are obtained. This corresponds to a yield of 69% t.q. or 89.7% calculated via the area fraction of the product peaks (both isomers) in the gas chromatogram (GC area %). The isomer ratio is 1.1:1.

The product is combined with further reaction discharges and fractionally distilled at a bath temperature of 180 to 215° C. and 0.25 mbar (Ti=125-160° C., transition temperature: 99-115° C.). The yield of the distillation is 90%, the isomer ratio of 16-methyloxacyclohexadecan-2-one and oxacycloheptadecan-2-one is approx. 1.2:1. The product is a clear, colorless liquid and has a purity of 94.5 GC area %.

1.-26. (canceled)

27. A process for the preparation of macrocyclic compounds of the general formula (I.a) or (I.b) 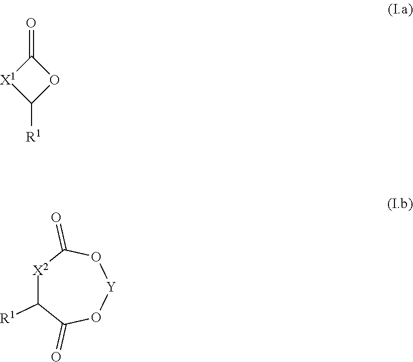
in which
X^{I} is an unbranched or branched C₄-C₃₀-alkylene group or an unbranched or branched C₄-C₃₀-alkenylene group, comprising 1, 2 or 3 double bonds,
X² is an unbranched or branched C₁-C₃₀-alkylene group or an unbranched or branched C₂-C₃₀-alkenylene group, comprising 1, 2 or 3 double bonds,
Y is an unbranched or branched C₂-C₁₀-alkylene group and
R¹ is hydrogen or an unbranched or branched C₁-C₁₀-alkyl group,
in which
a) providing at least one compound of the, general formula (II.a) or (II.b) 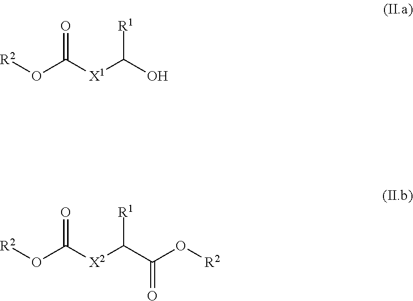
in which
X¹, X² and R¹ have the meanings given above and
R² is hydrogen or an unbranched or branched C₁-C₃₀-alkyl group,
b.1) the at least one compound (II.a) is reacted in the presence of at least one catalyst which is selected from metal alkoxides, and also in the presence of at least one polyether compound (PE) with a number-average molecular weight of at least 200 g/mol, to give a reaction mixture which comprises at least one macrocyclic compound of the general formula (I.a),
or
b.2) the at least one compound (II.b) is reacted in the presence of at least one catalyst which is selected from metal alkoxides, and also in the presence of at least one polyether compound (PE) with a number-average molecular weight of at least 200 g/mol, and additionally in the presence of at least one diol HO-Y-OH, where Y has the meaning given above, to give a reaction mixture which comprises at least one macrocyclic compound of the general formula (I.b),
where a product stream enriched in the macrocyclic compounds of the general formula (I.a) or (I.b) is removed by distillation from the reaction mixture obtained in step b.1) or b.2), and a bottom product enriched in the polyether compound (PE) and the catalyst is obtained, and where the at least one metal alkoxide catalyst used in step b.1) or b.2) is prepared by reacting at least one metal compound, selected from metal oxides, alkyl metal oxides, metal salts or metal alkoxides of the general formula M[O(C₁-C₄-alkyl)]ₘ, where m has the values 1, 2, 3 or 4, with at least one polyether compound (PE), where the preparation of the at least one metal alkoxide catalyst takes place before step b.1) or b.2), and where the at least one polyether compound (PE) is selected from compounds of the general formula (III)
R³—O—[Z—O]ₙ—R³ (III)
in which
Z is selected independently of the others from ethylene, 2-propylene, 1,3-propylene, 1, butylene, 2,3-butylene and 1,4-butylene,
n is an integer from 3 to 250 and
either one radical R³ is hydrogen and the other radical R³ is C₁-C₃₀-alkyl or
one radical R³ is hydrogen and the other radical R³ is —(C═O)—(C₁-C₃₀-alkyl) or
both radicals R³ are hydrogen or
both radicals R³ are —(C═O)—(C₁-C₃₀-alkyl).

28. The process according to claim 27, where the at least one catalyst used in step b.1) or b.2) has a boiling point at 5 mbar of more than 250° C.

29. The process according to claim 27, where, for the distillative removal of the product stream enriched in the compounds (I.a) or (I.b), at least one solvent (S) different from the polyether compound (PE) is added as entrainer to the reaction mixture obtained in step b.1) or b.2) and/or an inert gas stream is fed into the reaction mixture.

30. The process according to claim 27, where the distillative removal of the product stream enriched in the compounds (I.a) or (I.b) takes place after the reaction in step b.1) or b.2).

31. The process according to claim 27, where, in the compounds of the general formulae (I.a) and (I.b), the radical X¹ has 11 to 21 ring carbon atoms and the radicals X² and Y together have 9 to 19 ring carbon atoms.

32. The process according to claim 27, where, in the compounds of the general formulae (I.a), (I.b), (II.a) and (II.b),
R¹ is hydrogen or methyl,
X¹ is an unbranched C₁₂-C₁₆-alkylene group or an unbranched C₁₂-C₁₆-alkenylene group, comprising a double bond,
X² is an unbranched C₉-C₁₃-alkylene group or an unbranched C₉-C₁₃-alkenylene group, comprising a double bond,
Y is an unbranched C₂-C₄-alkylene group and
R² is hydrogen or an unbranched C₁-C₄-alkyl group,
where the radicals X² and Y together have 11 to 15 directly bridging carbon atoms.

33. The process according to claim 27, where the distillatively removed product stream enriched in the compounds (I.a) or (I.b) comprises at least some of the solvent (S) and optionally additionally some of the polyether compound (PE) and the product stream is subjected to a separation to give a fraction enriched in the solvent (S) and optionally in the polyether compound (PE) and a product fraction which comprises predominantly macrocyclic compounds of the general formula (I.a) or (I.b).

34. The process according to claim 33, where the product fraction is subjected to a further purification.

35. The process according to claim 33, where the fraction enriched in the solvent (S) and optionally in the polyether compound (PE) is returned again to the reaction in step b.1) or b.2).

36. The process according to claim 27, where the steps b.1) or b.2) and the distillative removal of the product stream enriched in the compounds (I.a) or (I.b) are carried out continuously.

37. The process according to claim 27, where either one radical R³ is hydrogen and the other radical R³ is C₁-C₁₀-alkyl or one radical R³ is hydrogen and the other radical R³ is —(C═O)—(C₁-C₁₀-alkyl) or both radicals R³ are hydrogen or both radicals R³ are —(C═O)—(C₁-C₁₀-alkyl).

38. The process according to claim 27, where one radical R³ is hydrogen and the other radical R³ is hydrogen or is C₁-C₁₀-alkyl or is —(C═O)—(C₁-C₁₀-alkyl).

39. The process according to claim 27, where the low-boiling components optionally forming during the preparation of the at least one metal alkoxide catalyst are removed by distillation.

40. The process according to claim 39, where, for the distillative removal of the low-boiling components optionally formed during the preparation of the at least one metal alkoxide catalyst, a stream of inert gas is used.

41. The process according to claim 27, where the metal of the metal oxide, alkyl metal oxide, metal salt or metal alkoxide M[O(C₁-C₄-alkyl)]ₘ, used for the preparation of the at least one metal alkoxide catalyst is selected from alkali metals, alkaline earth metals, transition metals of the 4th, 7th, 8th, 9th and 12th group, and metals and/or semi-metals of the 13th, 14th and 15th group of the Periodic Table of the Elements.

42. The process according to claim 27, where the preparation of the at least one metal alkoxide catalyst takes place in the absence of the feed materials (II.a), (II.b) and of the diol HO—Y—OH.

43. The process according to claim 27, where the bottom product enriched in the polyether compound (PE) and the catalyst is recycled to further reaction in step b.1) or b.2).

44. The process according to claim 27, where the at least one solvent (S) is selected from C₂-C₁₅-alkanols, glycerol, pentaerythritol, C₂-C₄-alkylene glycols and the mono- and di-(C₁-C₄-alkyl) ethers thereof, polyalkylene glycols different from the compounds PE and the mono- and dialkyl ethers thereof, aromatic hydrocarbons and mixtures thereof.

45. The process according to claim 27, where the provision of the compounds (II.a) or (II.b) comprises the following steps
a.1) providing of a C₆-C₂₂-carboxylic acid,
a.2) conversion of the C₆-C₂₂-carboxylic acid provided in step a.1) to omega- and/or (omega-1)-hydroxylated or omega-carboxylated C₆-C₂₂-carboxylic acids,
a.3) optionally the oxidation of the omega-hydroxylated C₆-C₂₂-carboxylic acids obtained in step a.2) to the corresponding omega-carboxylated C₆-C₂₂-carboxylic acids,
a.4) optionally the esterification of the omega- and/or (omega-1)-hydroxylated C₆-C₂₂-carboxylic acids from step a.2) or of the carboxylated C₆-C₂₂-carboxylic acids from steps a.2) or a.3) with unbranched or branched C₁-C₆-alkanols.

46. The process according to claim 45, where the hydroxylation or carboxylation in step a.2) is carried out by fermentation.

47. A compound (I.a) is (10Z,18S)-18-methyl-1-oxacyclooctadec-10-en-2-one, (10Z,18R)-18-methyl-1-oxacyclooctadec-10-en-2-one, (10E,18S)-18-methyl-1-oxacyclooctadec-10-en-2-one, or (10E,18R)-18-methyl-1-oxacyclooctadec-10-en-2-one.

48. A fragrance and/or flavoring comprising at least one of the compounds according to claim 47.

49. The fragrance and/or flavoring according to claim 48, where the at least one compound is a constituent of a composition which additionally comprises a carrier material.

50. The fragrance and/or flavoring according to claim 48, where the at least one compound is a constituent of a composition which additionally comprises a carrier material, where the composition is selected from detergents and cleaners, cosmetic preparations, fragrance containing hygiene articles, foods, food supplements, scent dispensers, perfumes, pharmaceutical preparations and crop protection agents.

51. A scent composition and/or a fragrance material comprising at least one of the compounds as claimed in claim 47 and a carrier material.

52. A method for imparting or altering an odor or taste of a composition, in which at least one of the compounds specified in claim 47 is added to the composition in an amount which imparts an odor or taste to the composition or alters the odor or taste of the composition.
